(19) United States
(12) Patent Application Publication
(10) Pub. No.: US 2026/0250394 A1
(43) Pub. Date: Aug. 27, 2026

(54) TREATMENT OF HEMATOLOGICAL MALIGNANCIES WITH ANTIBODIES INHIBITING GALECTIN-9

(71) Applicant: PURETECH LYT, INC., Boston, MA (US)

(72) Inventors: Aleksandra FILIPOVIC, Boston, MA (US); Christopher C. KORTH, Boston, MA (US); Adam N. GEORGE, Boston, MA (US); Julie Swartzman KROP, Boston, MA (US)

(73) Assignee: PURETECH LYT, INC., Boston, MA (US)

(21) Appl. No.: 18/879,335

(22) PCT Filed: Jun. 29, 2023
(86) PCT No.: PCT/US2023/069414

§ 371 Date: Dec. 27, 2024

Related U.S. Application Data
(60) Provisional application No. 63/356,840, filed on Jun. 29, 2022.
(60) Provisional application No. 63/394,507, filed on Aug. 2, 2022.
(60) Provisional application No. 63/394,506, filed on Aug. 2, 2022.
(60) Provisional application No. 63/425,986, filed on Nov. 16, 2022.

(51) Int. Cl. C07K 16/28; A61P 35/02

(52) U.S. Cl. CPC C07K 16/2851; A61P 35/02

(57) ABSTRACT

Disclosed herein are methods for treating a hematologic malignancy (e.g., acute myeloid leukemia (AML), myelodysplastic syndromes (MDS), or acute lymphoblastic leukemia (ALL)), using an anti-Galectin-9 antibody (e.g., G9.2-17(IgG4). In some embodiments, the anti-Galectin-9 antibody is used as a monotherapy.

### CROSS REFERENCE TO RELATED APPLICATIONS

This application claims the benefit of the filing dates of U.S. Provisional Application No. 63/356,840, filed Jun. 29, 2022, U.S. Provisional Application No. 63/394,506, filed Aug. 2, 2022, U.S. Provisional Application No. 63/394,507, filed Aug. 2, 2022, and U.S. Provisional Application No. 63/425,986, filed Nov. 16, 2022, the entire contents of each of which are incorporated by reference herein.

### REFERENCE TO SEQUENCE LISTING

The instant application contains a Sequence Listing which has been filed electronically in XML format and is hereby incorporated by reference in its entirety. Said XML copy, created on Jun. 29, 2023, is named 112174-0259-NP013WO00 and is 40,880 15 bytes in size.

### BACKGROUND OF INVENTION

Galectin-9 is a tandem-repeat lectin consisting of two carbohydrate recognition domains (CRDs) and was discovered and described for the first time in 1997 in patients suffering from Hodgkin's lymphoma (HL) (Tureci et al., J. Biol. Chem. 1997, 272, 6416-6422). Three isoforms exist and can be located within the cell or extracellularly. Elevated Galectin-9 levels have been in observed a wide range of cancers, including melanoma, Hodgkin's lymphoma, hepatocellular, pancreatic, gastric, colon and clear cell renal cell cancers (Wdowiak et al. Int. J. Mol. Sci. 2018, 19, 210). In renal cancer, patients with high Galectin-9 expression showed more advanced progression of the disease with larger tumor size (Kawashima et al.; BJU Int. 2014; 113:320-332). In melanoma, Galectin-9 was expressed in 57% of tumors and was significantly increased in the plasma of patients with advanced melanoma compared to healthy controls (Enninga et al., Melanoma Res. 2016 October; 26(5): 429-441). A number of studies have shown utility for Galectin-9 as a prognostic marker, and more recently as a potential new drug target (Enninga et al., 2016; Kawashima et al. BJU Int 2014; 113: 320-332; Kageshita et al., Int J Cancer. 2002 Jun. 20; 99(6):809-16, and references therein).

Galectin-9 has been described to play an important role in in a number of cellular processes such as adhesion, cancer cell aggregation, apoptosis, and chemotaxis. Recent studies have shown a role for Galectin-9 in immune modulation in support of the tumor, e.g., through negative regulation of Th1 type responses, Th2 polarization and polarization of macrophages to the M2 phenotype. This work also includes studies that have shown that Galectin-9 participates in direct inactivation of T cells through interactions with the T-cell immunoglobulin and mucin protein 3 (TIM-3) receptor (Dardalhon et al., J Immunol., 2010, 185, 1383-1392; Sanchez-Fueyo et al., Nat Immunol., 2003, 4, 1093-1101).

Galectin-9 has also been found to play a role in polarizing T cell differentiation into tumor suppressive phenotypes), as well as promoting tolerogenic macrophage programming and adaptive immune suppression (Daley et al., Nat Med., 2017, 23, 556-567). In mouse models of pancreatic ductal adenocarcinoma (PDAC), blockade of the checkpoint interaction between Galectin-9 and the receptor Dectin-1 found on innate immune cells in the tumor microenvironment (TME) has been shown to increase anti-tumor immune responses in the TME and to slow tumor progression (Daley et al., Nat Med., 2017, 23, 556-567). Galectin-9 also has been found to bind to CD206, a surface marker of M2 type macrophages, resulting in a reduced secretion of CVL22 (MDC), a macrophage derived chemokine which has been associated with longer survival and lower recurrence risk in lung cancer (Enninga et al, J Pathol. 2018 August; 245(4):468-477).

### SUMMARY OF INVENTION

The present disclosure is based, at least in part, on the development of treatment regimen for hematological malignancies such as acute myeloid leukemia (AML), myelodysplastic syndromes (MDS), or acute lymphoblastic leukemia (ALL).

Accordingly, provided herein is a method for treating a hematologic malignancy, comprising administering to a subject in need thereof a pharmaceutical composition comprising an antibody that binds human galectin-9 (anti-Gal9 antibody), wherein the anti-Gal9 antibody is administered to the subject at a dose of about 2 mg/kg to about 32 mg/kg. In some embodiments, the anti-Gal9 antibody is administered to the subject once every week to once every 6 weeks (e.g., once every week, once every two weeks, once every three weeks, once every 4 weeks, once every five weeks, once every six weeks). For example, the anti-Gal9 antibody may be administered to the subject at 2 mg/kg, 4 mg/kg, 6.3 mg/kg, 7.5 mg/kg, 10 mg/kg, 12 mg/kg, 16 mg/kg, or 32 mg/kg once every week to once every six weeks (e.g., once every week, once every two weeks, once every three weeks, once every 4 weeks, once every five weeks, once every six weeks). In some embodiments, the anti-Gal9 antibody is administered once every week. In some embodiments, the anti-Gal9 antibody is administered once every two weeks. In some embodiments, any of the anti-Galectin-9 antibodies disclosed herein may be administered to the subject by intravenous infusion.

In some embodiments, the anti-Gal9 antibody may comprise: (a) a light chain comprising alight chain variable region (V_{L}), which comprises a light chain (LC) complementarity determining region 1 (CDR1) comprising the amino acid sequence of SEQ ID NO: 1, a LC complementarity determining region 2 (CDR2) comprising the amino acid sequence of SEQ ID NO: 2, and a LC complementarity determining region 3 (CDR3) comprising the amino acid sequence of SEQ ID NO: 3 and (b) a heavy chain comprising a heavy chain variable region (V_{H}), which comprises a heavy chain (HC) complementarity determining region 1 (CDR1) comprising the amino acid sequence of SEQ ID NO: 4, a HC complementarity determining region 2 (CDR2) comprising the amino acid sequence of SEQ ID NO: 5, and a HC complementarity determining region 3 (CDR3) comprising the amino acid sequence of SEQ ID NO: 6. In some examples, the anti-Gal9 antibody comprises the V_{L}, which comprises the amino acid sequence of SEQ ID NO:8, and the V_{H}, which comprises the amino acid sequence of SEQ ID NO: 7.

Any of the anti-Gal9 antibodies for use in the method disclosed herein may be an IgG1 molecule. Alternatively, the antibody may be an IgG4 molecule. In some embodiments, the anti-Gal9 antibody can be an IgG4 molecule (e.g., a human IgG4 molecule) comprising a modified Fc region relative to the wild-type IgG4 counterpart (e.g., the wild-type human IgG4 counterpart). In some examples, the modified Fc region comprises the amino acid sequence of SEQ ID NO: 14. In one example, the anti-Gal9 antibody comprises a heavy chain, which comprises the amino acid sequence of SEQ ID NO: 19, and a light chain, which comprises the amino acid sequence of SEQ ID NO: 15. Such an anti-Gal9 antibody may be G9.2-17 (IgG4).

Any of the anti-Gal9 antibody disclosed herein (e.g., G9.2-17 (IgG4)) may be administered to the subject at a dose of 2 mg/kg, 4 mg/kg, 6.3 mg/kg, 7.5 mg/kg, 10 mg/kg, 12 mg/kg, 16 mg/kg, or 32 mg/kg. In some embodiments, the anti-Gal9 antibody is administered to the subject once every week to once every 6 weeks (e.g., once every week, once every two weeks, once every three weeks, once every 4 weeks, once every five weeks, once every six weeks). For example, the anti-Gal9 antibody may be administered to the subject at 2 mg/kg, 4 mg/kg, 6.3 mg/kg, 7.5 mg/kg, 10 mg/kg, 12 mg/kg, 16 mg/kg, or 32 mg/kg once every week to once every six weeks (e.g., once every week, once every two weeks, once every three weeks, once every 4 weeks, once every five weeks, once every six weeks). In some embodiments, the anti-Gal9 antibody is administered once every week. In some embodiments, the anti-Gal9 antibody is administered once every two weeks to once every four weeks. In some examples, the anti-Galectin-9 antibody may be administered to the subject once every two weeks. In specific embodiments, the anti-Galectin-9 antibody (e.g., G9.2-17 (IgG4)) is administered to the subject at a dose of 6.3 mg/kg, 7.5 mg/kg, 10 mg/kg, 12 mg/kg, or 16 mg/kg once every week. In specific embodiments, the anti-Galectin-9 antibody (e.g., G9.2-17 (IgG4)) is administered to the subject at a dose of 6.3 mg/kg, 7.5 mg/kg, 10 mg/kg, 12 mg/kg, or 16 mg/kg once every two weeks to once every four weeks (e.g., once every two weeks).

Alternatively, the anti-Gal-9 antibody such as G9.2-17 (IgG4) may be administered to a subject at a dose of about 410 mg to about 2056 mg (e.g., about 410 to about 650 mg, about 410 to about 1040 mg, or about 410 to about 1280 mg once every 2-6 weeks (e.g., every 2-4 weeks), for example, once every 2 weeks, once every 3 weeks, once every 4 weeks, once every 5 weeks, or once every 6 weeks. In some examples, the anti-Gal-9 antibody is administered to a subject at a dose of about 410 mg to about 505 mg (e.g., about 410 to about 450 mg or about 410 to about 480) once every 2-6 weeks (e.g., every 2-4 weeks), for example, once every 2 weeks, once every 3 weeks, once every 4 weeks, once every 5 weeks, or once every 6 weeks. In other examples, the anti-Gal-9 antibody is administered to a subject at a dose of about 650 mg to about 800 mg (e.g., about 650 to about 700 mg) once every 2-6 weeks (e.g., every 2-4 weeks), for example, once every 2 weeks, once every 3 weeks, once every 4 weeks, once every 5 weeks, or once every 6 weeks. In yet other examples, the anti-Gal-9 antibody is administered to a subject at a dose of about 1040 mg to about 1280 mg (e.g., about 1040 to about 1120 mg once every 2-6 weeks (e.g., every 2-4 weeks), for example, once every 2 weeks, once every 3 weeks, once every 4 weeks, once every 5 weeks, or once every 6 weeks. In yet other examples, the anti-Gal-9 antibody is administered to a subject at a dose of about 2080 mg to about 2560 mg (e.g., about 2080 to about 2400 mg) once every 2-6 weeks (e.g., every 2-4 weeks), for example, once every 2 weeks, once every 3 weeks, once every 4 weeks, once every 5 weeks, or once every 6 weeks.

In other embodiments, any of the anti-Gal9 antibody disclosed herein (e.g., G9.2-17 (IgG4)) may be administered to the subject at a dose of 2 mg/kg, 4 mg/kg, 6.3 mg/kg, 7.5 mg/kg, 10 mg/kg, 12 mg/kg, 16 mg/kg, or 32 mg/kg once every week. In specific embodiments, the anti-Galectin-9 antibody (e.g., G9.2-17 (IgG4)) is administered to the subject at a dose of 4 mg/kg, 6.3 mg/kg, 7.5 mg/kg, 10 mg/kg, 12 mg/kg or 16 mg/kg, once every week.

Alternatively, the anti-Gal-9 antibody such as G9.2-17 (IgG4) may be administered to a subject at a dose of about 410 mg to about 2560 (e.g., about 410 mg to about 1040 mg or about 410 mg to about 650 mg) once every week. In some examples, the anti-Gal-9 antibody is administered to a subject at a dose of about 410 mg to about 505 (e.g., about 410 mg to about 450 mg) once every week. In other examples, the anti-Gal-9 antibody is administered to a subject at a dose of about 650 mg to about 800 mg (e.g., about 650 to about 700 mg) once every week. In yet other examples, the anti-Gal-9 antibody is administered to a subject at a dose of about 1040 mg to about 1280 (e.g., about 1040 mg to about 1120 mg) once every week. In yet other examples, the anti-Gal-9 antibody is administered to a subject at a dose of about 2080 mg to about 2560 mg (e.g., about 2080 to about 2400 mg) once every week.

In one example, the anti-Gal9 antibody disclosed herein (e.g., G9.2-17 (IgG4)) may be administered to the subject at a dose of 2 mg/kg once every week. In another example, the anti-Gal9 antibody disclosed herein (e.g., G9.2-17 (IgG4)) may be administered to the subject at a dose of 4 mg/kg once every week. In still another example, the anti-Gal9 antibody disclosed herein (e.g., G9.2-17 (IgG4)) may be administered to the subject at a dose of 7.5 mg/kg once every week. In yet another example, the anti-Gal9 antibody disclosed herein (e.g., G9.2-17 (IgG4)) may be administered to the subject at a dose of 10.0 mg/kg once every week. In yet another example, the anti-Gal9 antibody disclosed herein (e.g., G9.2-17 (IgG4)) may be administered to the subject at a dose of 12.0 mg/kg once every week. In another example, the anti-Gal9 antibody disclosed herein (e.g., G9.2-17 (IgG4)) may be administered to the subject at a dose of 16.0 mg/kg once every week.

In some embodiments, the subject for treatment by any of the methods disclosed herein is a human patient having a leukemia or lymphoma. In specific examples, the human patient has acute myeloid leukemia (AML), myelodysplastic syndromes (MDS), or acute lymphoblastic leukemia (ALL, such as T-cell ALL or B-cell ALL). Alternatively or in addition, the hematologic malignancy is refractory and/or relapsed. In some instances, the human patient has relapsed and/or refractory AML post at least one line of prior therapy. In other instances, the human patient has relapsed and/or refractory MDS post at least one line of prior therapy. Alternatively or in addition, the human patient does not have acute promyelocytic leukemia (APL).

In some embodiments, the human patient has undergone or is undergoing a chemotherapy for treating the hematologic malignancy. In other embodiments, the human patient has not undergone or is not undergoing a chemotherapy for treating the hematologic malignancy.

Any of the methods disclosed herein may effectively reduce or eliminate Gal9+ hematological cancer cells, for example, leukemia or lymphoma cells. Alternatively or in addition, the method disclosed herein may effectively modulate immune responses targeting the cancer cells.

In some instances, any of the methods disclosed herein comprises at least one cycle of the treatment that consists of 28 days, during which the anti-Gal9 antibody is administered to the subject once every week. For example, the treatment may comprise one cycle, two cycles, three cycles, four cycles, five cycles, six cycles, or more.

In some instances, the method disclosed herein comprises the anti-Gal9 antibody as the only active agent for treating the hematologic malignancy (monotherapy).

Any of the methods disclosed herein may further comprise monitoring occurrence of an adverse event in the course of the treatment. In some instances, the dose of the anti-Gal9 antibody may be reduced when the adverse event occurs.

Also provided herein are anti-Gal9 antibodies (e.g., G9.2-17(IgG4)) for use in treating a hematological cancer (e.g., those disclosed herein) in any of the methods disclosed herein and uses of such anti-Gal9 antibodies for manufacturing a medicament for use in the treatment method disclosed herein.

The details of one or more embodiments of the invention are set forth in the description below. Other features or advantages of the present invention are to be apparent from the following drawing and detailed description of several embodiments, and also from the appended claims.

### BRIEF DESCRIPTION OF THE DRAWINGS

FIG. 1 is a diagram depicting a study schematic of using anti-Gal9 antibody G9.2-17(IgG4) for treating hematologic malignancy such as AML and high risk MDS. RP2D=recommended phase 2 dose. PK=pharmacokinetics. PD=pharmacodynamics.

FIG. 2 is a diagram depicting clinical trial schedule of assessment. Phase 1 enrolls up to 40 patients. Each patient will undergo clinical visit for pre-cycle 1 dose 1 administration (preC1), as well as for drug administration C1D1 (cycle 1 day 1 etc.), and QW thereafter. Note that MRD will be assessed form bone marrow and blood, whenever the bone marrow aspirate/biopsy is possible concomitantly. Bone marrow analyses will post month 4 be done once every 3 months provided the patient is still on study drug. Blood MRD assessment will continue monthly.

FIG. 3 is a diagram showing markers for examination.

### DETAILED DESCRIPTION OF INVENTION

Galectin-9, a tandem-repeat lectin, is a beta-galactoside-binding protein, which has been shown to have a role in modulating cell-cell and cell-matrix interactions. It is found to be strongly overexpressed in Hodgkin's disease tissue and in other pathologic states. It may also be found circulating in the tumor microenvironment (TME).

Galectin-9 is found to interact with TIM-3, a type I cell surface glycoprotein expressed on the surface of leukemic stem cells in all varieties of acute myeloid leukemia (except for M3 (acute promyelocytic leukemia)), but not expressed in normal human hematopoietic stem cells (HSCs). TIM-3 signaling resulting from Galectin-9 ligation has been found to have a pleiotropic effect on immune cells, inducing apoptosis in Th1 cells (Zhu et al., Nat Immunol., 2005, 6: 1245-1252) and stimulating the secretion of tumor necrosis factor-a (TNF-a), leading to the maturation of monocytes into dendritic cells, resulting in inflammation by innate immunity (Kuchroo et al., Nat Rev Immunol., 2008, 8:577-580). Further Galectin-9/TIM-3 signaling has been found to co-activate NF-KB and catenin signaling, two pathways that promote LSC self-renewal (Kikushige et al., Cell Stem Cell, 2015, 17(3):341-352). An antiGalectin-9 antibody that interferes with Galectin-9/TIM-3 binding could have a therapeutic effect, especially with respect to leukemia and other hematological malignancies.

Further, Galectin-9 is a pleiotropic immune modulator affecting numerous cell types of innate and adaptive immunity. Gal-9 promotes inflammation, induces M2 macrophages and monocyte-derived dendritic cells (mDC), promotes differentiation of regulatory T cells, and suppresses NK cell killing ability. There are multiple transcriptional and functional changes induced by galectin-9 independent of Tim-3. Gal9 expression is also found to be higher in AML patients who fail chemotherapy. See, e.g., OncoImmunology, 5(7):00-00 DOI: 10.1080/2162402X.2016.1195535. Anti-Gal9 antibody was found to significantly inhibits reconstitution of AML as well as LSCs. Kikushige et al., Cell Stem Cell 17(3):341-352 (2015).

The anti-Gal9 antibodies disclosed herein, such as G9.2-17 (IgG4), can directly kill Gal9+ hematologic cancer cells via induction of cytotoxicity. Further, Galectin-9 is also found to affect multiple pathways of immunosuppression. The immunology effect enables an anti-Gal9 antibody such as G9.2-17(IgG4) as a potential single-agent treatment efficacy.

Accordingly, provided herein are methods of using anti-Galectin-9 antibodies, e.g., G9.2-17 (IgG4), for treating a hematological malignancy (e.g., acute myeloid leukemia (AML), myelodysplastic syndromes (MDS), or acute lymphoblastic leukemia (ALL, such as T-cell ALL or B-cell ALL)) at a dose of about 2 mg/kg to about 32 mg/kg (or about 130 mg to about 2,560 mg) once every week to once every 6 weeks (e.g., once every week to once every 4 weeks), for example, once every week or once every 2 weeks.

### I. Antibodies Binding to Galectin-9

The present disclosure provides anti-Galectin-9 antibodies, such as G9.2-17 and functional variants thereof, for use in the treatment methods disclosed herein.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody”, e.g., anti-Galectin-9 antibody, encompasses not only intact (e.g., full-length) polyclonal or monoclonal antibodies, but also antigen-binding fragments thereof (such as Fab, Fab′, F(ab′)₂, Fv), single chain (scFv), mutants thereof, fusion proteins comprising an antibody portion, humanized antibodies, chimeric antibodies, diabodies, nanobodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-Galectin-9 antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, the EU definition, the “Contact” numbering scheme, the IMGT” numbering scheme, the “AHo” numbering scheme, and/or the contact definition, all of which are well known in the art. See, e.g., Kabat, E. A., et al. (1991) Sequences of proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; Edelman et al., Proc Natl Acad Sci USA. 1969 May; 63(1):78-85; and Almagro, J. Mol. Recognit. 17:132-143 (2004); MacCallum et al., J. Mol. Biol. 262:732-745 (1996), Lefranc M P et al., Dev Comp Immunol, 2003 January; 27(1):55-77; and Honegger A and Pluckthun A, J Mol Biol, 2001 Jun. 8; 309(3):657-70. See also hgmp.mrc.ac.uk and bioinf.org.uk/abs).

In some embodiments, the anti-Galectin-9 antibody described herein is a full-length antibody, which contains two heavy chains and two light chains, each including a variable domain and a constant domain. Alternatively, the anti-Galectin-9 antibody can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

Any of the antibodies described herein, e.g., anti-Galectin-9 antibody, can be either monoclonal or polyclonal. A “monoclonal antibody” refers to a homogenous antibody population and a “polyclonal antibody” refers to a heterogeneous antibody population. These two terms do not limit the source of an antibody or the manner, in which it is made.

Reference antibody G9.2-17 refers to an antibody capable of binding to human Galectin-9 and comprises a heavy chain variable region of SEQ ID NO: 7 and a light chain variable domain of SEQ ID NO: 8, both of which are provided below. In some embodiments, the anti-Galectin-9 antibody for use in the methods disclosed herein is the G9.2-17 antibody. In some embodiments, the anti-Galectin-9 antibody for use in the methods disclosed herein is an antibody having the same heavy chain complementarity determining regions (CDRs) as reference antibody G9.2-17 and/or the same light chain complementarity determining regions as reference antibody G9.2-17. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/).

The heavy and light chain CDRs of reference antibody G9.2-17 is provided in Table 1 below (determined using the Kabat methodology):

TABLE 1
Heavy and Light Chain CDRs of G9.2-17

| | | | |
|---|---|---|---|
| G9.2-17 | V_{L} CDR1 | RASQSVSSA | SEQ ID NO: 1 |
| | | VA | |
| | V_{L} CDR2 | SASSLYS | SEQ ID NO: 2 |
| | V_{L} CDR3 | QQSSTDPIT | SEQ ID NO: 3 |
| | V_{H} CDR1 | FTVSSSSIH | SEQ ID NO: 4 |
| | V_{H} CDR2 | YISSSSGYT | SEQ ID NO: 5 |
| | | YYADSVKG | |
| | V_{H} CDR3 | YWSYPSWWP | SEQ ID NO: 6 |
| | | YRGMDY | |

In some examples, the anti-Galectin-9 antibody for use in the methods disclosed herein may comprise (following the Kabat scheme) a heavy chain complementarity determining region 1 (CDR1) set forth as SEQ ID NO: 4, a heavy chain complementarity determining region 2 (CDR2) set forth as SEQ ID NO: 5, and a heavy chain complementarity determining region 3 (CDR3) set forth as SEQ ID NO: 6 and/or may comprise a light chain complementarity determining region 1 (CDR1) set forth as SEQ ID NO: 1, a light chain complementarity determining region 2 (CDR2) set forth as SEQ ID NO: 2, and a light chain complementarity determining region 3 (CDR3) set forth as SEQ ID NO: 3. The anti-Galectin-9 antibody, including the reference antibody G9.2-17, can be in any format as disclosed herein, for example, a full-length antibody or a Fab. The term “G9.2-17(IgG4)” used herein refers to a G9.2-17 antibody which is an IgG4 molecule. Likewise, the term “G9.2-17 (Fab)” refers to a G9.2-17 antibody, which is a Fab molecule.

In some embodiments, the anti-Galectin-9 antibody or binding portion thereof comprises heavy and light chain variable regions, wherein the light chain variable region CDR1, CDR2, and CDR3 amino acid sequences have at least 80% (e.g., 80%, 85%, 90%, 91%, 92%, 93%, 94%, 95%, 96%, 97%, 98%, or 99% and any increment therein) sequence identity to the light chain variable region CDR1, CDR2, and CDR3 amino acid sequences set forth in SEQ ID NOs: 1, 2, and 3, respectively. In some embodiments, the anti-Galectin-9 antibody or binding portion thereof comprises heavy and light chain variable regions, wherein the heavy chain variable region CDR1, CDR2, and CDR3 amino acid sequences have at least 80% (e.g., 80%, 85%, 90%, 91%, 92%, 93%, 94%, 95%, 96%, 97%, 98%, or 99% and any increment therein) sequence identity to the heavy chain variable region CDR1, CDR2, and CDR3 amino acid sequences set forth in SEQ ID NO: 4, 5, and 6, respectively.

Additional Galectin-9 antibodies, e.g., which bind to the CRD1 and/or CRD2 region of Galectin-9 are described in U.S. Pat. No. 10,344,091 and co-owned, co-pending U.S. patent application Ser. No. 16/173,970 and WO 2020/198390, the relevant disclosures of each of which are incorporated by reference for the subject matter and purposes referenced herein.

In some embodiments, the anti-Galectin-9 antibody disclosed herein comprises light chain CDRs that have at least 80% (e.g., 80%, 85%, 90%, 91%, 92%, 93%, 94%, 95%, 96%, 97%, 98%, or 99% and any increment therein) sequence identity, individually or collectively, as compared with the corresponding V_{L} CDRs of reference antibody G9.2-17. Alternatively or in addition, in some embodiments, the anti-Galectin-9 antibody comprises heavy chain CDRs that have at least 80% (e.g., 80%, 85%, 90%, 91%, 92%, 93%, 94%, 95%, 96%, 97%, 98%, or 99% and any increment therein) sequence identity, individually or collectively, as compared with the corresponding V_{H} CDRs of reference antibody G9.2-17.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In other embodiments, the anti-Galectin-9 antibody described herein comprises a V_{H} that comprises the HC CDR1, HC CDR2, and HC CDR3, which collectively contain up to 8 amino acid residue variations (8, 7, 6, 5, 4, 3, 2, or 1 variation(s), including additions, deletions, and/or substitutions) relative to the HC CDR1, HC CDR2, and HC CDR3 of reference antibody G9.2-17. Alternatively or in addition, in some embodiments, the anti-Galectin-9 antibody described herein comprises a V_{H} that comprises the LC CDR1, LC CDR2, and LC CDR3, which collectively contain up to 8 amino acid residue variations (8, 7, 6, 5, 4, 3, 2, or 1 variations(s) including additions, deletions, and/or substitutions) relative to the LC CDR1, LC CDR2, and LC CDR3 of reference antibody G9.2-17.

In one example, the amino acid residue variations are conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-Galectin-9 antibodies disclosed herein, having the heavy chain CDRs disclosed herein, contains framework regions derived from a subclass of germline V_{H} fragment. Such germline V_{H} regions are well known in the art. See, e.g., the IMGT database (www.imgt.org) or at www.vbase2.org/vbstat.php. Examples include the IGHV1 subfamily (e.g., IGHV1-2, IGHV1-3, IGHV1-8, IGHV1-18, IGHV1-24, IGHV1-45, IGHV1-46, IGHV1-58, and IGHV1-69), the IGHV2 subfamily (e.g., IGHV2-5, IGHV2-26, and IGHV2-70), the IGHV3 subfamily (e.g., IGHV3-7, IGHV3-9, IGHV3-11, IGHV3-13, IGHV3-15, IGHV3-20, IGHV3-21, IGHV3-23, IGHV3-30, IGHV3-33, IGHV3-43, IGHV3-48, IGHV3-49, IGHV3-53, IGHV3-64, IGHV3-66, IGHV3-72, and IGHV3-73, IGHV3-74), the IGHV4 subfamily (e.g., IGHV4-4, IGHV4-28, IGHV4-31, IGHV4-34, IGHV4-39, IGHV4-59, IGHV4-61, and IGHV4-B), the IGHV subfamily (e.g., IGHV5-51, or IGHV6-1), and the IGHV7 subfamily (e.g., IGHV7-4-1).

Alternatively, or in addition, in some embodiments, the anti-Galectin-9 antibody, having the light chain CDRs disclosed herein, contains framework regions derived from a germline Vx fragment. Examples include an IGKV1 framework (e.g., IGKV1-05, IGKV1-12, IGKV1-27, IGKV1-33, or IGKV1-39), an IGKV2 framework (e.g., IGKV2-28), an IGKV3 framework (e.g., IGKV3-11, IGKV3-15, or IGKV3-20), and an IGKV4 framework (e.g., IGKV4-1). In other instances, the anti-Galectin-9 antibody comprises a light chain variable region that contains a framework derived from a germline Vλ fragment. Examples include an IGλ1 framework (e.g., IGλV1-36, IGλV1-40, IGλV1-44, IGλV1-47, IGλV1-51), an IGλ2 framework (e.g., IGλV2-8, IGλV2-11, IGλV2-14, IGλV2-18, IGλV2-23,), an IGλ3 framework (e.g., IGλV3-1, IGλV3-9, IGλV3-10, IGλV3-12, IGλV3-16, IGλV3-19, IGλV3-21, IGλV3-25, IGλV3-27,), an IGλ4 framework (e.g., IGλV4-3, IGλV4-60, IGλV4-69,), an IGλ5 framework (e.g., IGλV5-39, IGλV5-45), an IGλ6 framework (e.g., IGλV6-57,), an IGλ7 framework (e.g., IGλV7-43, IGλV7-46), an IGλ8 framework (e.g., IGλV8-61), an IGλ9 framework (e.g., IGλV9-49), or an IGλ10 framework (e.g., IGλV10-54).

In some embodiments, the anti-Galectin-9 antibody for use in the method disclosed herein can be an antibody having the same heavy chain variable region (V_{H}) and/or the same light chain variable region (V_{L}) as reference antibody G9.2-17, the V_{H} and V_{L} region amino acid sequences are provided below:

V_{H}:
(SEQ ID NO: 7)
EVQLVESGGGLVQPGGSLRLSCAASGFTVSSSSIHWVRQAPGKGLEW
VAYISSSSGYTYYADSVKGRFTISADTSKNTAYLQMNSLRAEDTAVY
YCARYWSYPSWWPYRGMDYWGQGTLVTVSS
V_{L}:
(SEQ ID NO: 8)
DIQMTQSPSSLSASVGDRVTITCRASQSVSSAVAWYQQKPGKAPKLL
IYSASSLYSGVPSRFSGSRSGTDFTLTISSLQPEDFATYYCQQSSTD
PITFGQGTKVEIKR

In some embodiments, the anti-Galectin-9 antibody has at least 80% sequence identity (e.g., 80%, 85%, 90%, 91%, 92%, 93%, 94%, 95%, 96%, 97%, 98%, or 99% identity) to the heavy chain variable region of SEQ ID NO: 7. Alternatively or in addition, the anti-Galectin-9 antibody has at least 80% sequence identity (e.g., 80%, 85%, 90%, 91%, 92%, 93%, 94%, 95%, 96%, 97%, 98%, or 99% identity) to the light chain variable region of SEQ ID NO: 8.

In some instances, the anti-Galectin-9 antibody disclosed herein is a functional variant of reference antibody G9.2-17. A functional variant can be structurally similar as the reference antibody (e.g., comprising the limited number of amino acid residue variations in one or more of the heavy chain and/or light chain CDRs as G9.2-17 as disclosed herein, or the sequence identity relative to the heavy chain and/or light chain CDRs of G9.2-17, or the V_{H} and/or V_{L} of G9.2-17 as disclosed herein) with substantially similar binding affinity (e.g., having a KD value in the same order) to human Galectin-9.

In some embodiments, the anti-Galectin-9 antibody as described herein can bind and inhibit the activity of Galectin-9 by at least 20% (e.g., 31%, 35%, 40%, 45%, 50%, 60%, 70%, 80%, 90%, 95% or greater, including any increment therein). The apparent inhibition constant (Ki^{app} or K_{i,app}), which provides a measure of inhibitor potency, is related to the concentration of inhibitor required to reduce enzyme activity and is not dependent on enzyme concentrations. The inhibitory activity of an anti-Galectin-9 antibody described herein can be determined by routine methods known in the art.

The K_{i,}^{app} value of an antibody may be determined by measuring the inhibitory effect of different concentrations of the antibody on the extent of the reaction (e.g., enzyme activity); fitting the change in pseudo-first order rate constant (v) as a function of inhibitor concentration to the modified Morrison equation (Equation 1) yields an estimate of the apparent Ki value. For a competitive inhibitor, the Ki^{app} can be obtained from the y-intercept extracted from a linear regression analysis of a plot of K_{i,}^{app} versus substrate concentration.

$$v = A \cdot \frac{( [ E ] - [ I ] - {K}_{i}^{app} ) + \sqrt{{( [ E ] - [ I ] - {K}_{i}^{app} )}^{2} + 4 [ E ] \cdot } {K}_{i}^{app}}{2} \qquad ( Equation 1 )$$

Where A is equivalent to vₒ/E, the initial velocity (vₒ) of the enzymatic reaction in the absence of inhibitor (I) divided by the total enzyme concentration (E). In some embodiments, the anti-Galectin-9 antibody described herein has a Ki^{app} value of 1000, 900, 800, 700, 600, 500, 400, 300, 200, 100, 50, 40, 30, 20, 19, 18, 17, 16, 15, 14, 13, 12, 11, 10, 9, 8, 7, 6, 5 pM or less for the target antigen or antigen epitope. In some embodiments, the anti-Galectin-9 antibody has a lower Ki^{app} for a first target (e.g., the CRD2 of Galectin-9) relative to a second target (e.g., CRD1 of the Galectin-9). Differences in Ki^{app} (e.g., for specificity or other comparisons) can be at least 1.5, 2, 3, 4, 5, 10, 15, 20, 37.5, 50, 70, 80, 91, 100, 500, 1000, 10,000 or 105 fold. In some examples, the anti-Galectin-9 antibody inhibits a first antigen (e.g., a first protein in a first conformation or mimic thereof) greater relative to a second antigen (e.g., the same first protein in a second conformation or mimic thereof; or a second protein). In some embodiments, any of the anti-Galectin-9 antibodies is further affinity matured to reduce the Ki^{app} of the antibody to the target antigen or antigenic epitope thereof.

In some embodiments, an anti-Galectin-9 antibody as described herein has a suitable binding affinity for the target antigen (e.g., Galectin-9) or antigenic epitopes thereof. As used herein, “binding affinity” refers to the apparent association constant or K_{A}. The K_{A} is the reciprocal of the dissociation constant (K_{D}). The anti-Galectin-9 antibody described herein may have a binding affinity (K_{D}) of at least 10⁻⁵, 10⁻⁶, 10⁻⁷, 10⁻⁸, 10⁻⁹, 10⁻¹⁰ M, or lower for the target antigen or antigenic epitope. An increased binding affinity corresponds to a decreased K_{D}. Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20).

These techniques can be used to measure the concentration of bound binding protein as a function of target protein concentration. Under certain conditions, the fractional concentration of bound binding protein (┌Bound┐/└Total┐) is generally related to the concentration of total target protein (└Target┘) by the following equation:

$$⌊ Bound ⌋ / ⌊ Total ⌋ = ⌊ Target ⌋ / ( Kd + ⌊ Target ⌋ )$$

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay. In some cases, the in vitro binding assay is indicative of in vivo activity. In other cases, the in vitro binding assay is not necessarily indicative of in vivo activity. In some cases, tight binding is beneficial, but in other cases tight binding is not as desirable in vivo, and an antibody with lower binding affinity is more desirable.

In some embodiments, the heavy chain of any of any of the anti-Galectin-9 antibodies as described herein further comprise a heavy chain constant region (CH) or a portion thereof (e.g., CH1, CH2, CH3, or a combination thereof). The heavy chain constant region can be of any suitable origin, e.g., human, mouse, rat, or rabbit. In one specific example, the heavy chain constant region is from a human IgG (a gamma heavy chain) of any IgG subfamily as described herein.

In some embodiments, the heavy chain constant region of the antibodies described herein comprise a single domain (e.g., CH1, CH2, or CH3) or a combination of any of the single domains, of a constant region (e.g., SEQ ID NO: 4, 5, 6). In some embodiments, the light chain constant region of the antibodies described herein comprise a single domain (e.g., CL), of a constant region. Exemplary light and heavy chain sequences are listed below. Exemplary light and heavy chain sequences are listed below. The hIgG1 LALA sequence includes two mutations, L234A and L235A (EU numbering), which suppress FcgR binding as well as a P329G mutation (EU numbering) to abolish complement C1q binding, thus abolishing all immune effector functions. The hIgG4 Fab Arm Exchange Mutant sequence includes a mutation to suppress Fab Arm Exchange (S228P; EU numbering). An IL2 signal sequence (MYRMQLLSCIALSLALVTNS; SEQ ID NO: 9) can be located N-terminally of the variable region. It is used in expression vectors, which is cleaved during secretion and thus not in the mature antibody molecule. The mature protein (after secretion) starts with “EVQ” for the heavy chain and “DIM” for the light chain. Amino acid sequences of exemplary heavy chain constant regions are provided below:

hIgG1 Heavy Chain Constant Region
(SEQ ID NO: 10)
ASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSV
LTVLHQDWLNGKEYKCKVSNKALPAPIEKTISKAKGQPREPQVYTLP
PSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG
K*
hIgG1 LALA Heavy Chain Constant Region
(SEQ ID NO: 12)
ASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKV
DKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSV
LTVLHQDWLNGKEYKCKVSNKALGAPIEKTISKAKGQPREPQVYTLP
PSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG
K*
hIgG4 Heavy Chain Constant Region
(SEQ ID NO: 13)
ASTKGPSVFPLAPCSRSTSESTAALGCLVKDYFPEPVTVSWNSGALT
SGVHTFPAVLQSSGLYSLSSVVTVPSSSLGTKTYTCNVDHKPSNTKV
DKRVESKYGPPCPSCPAPEFLGGPSVFLFPPKPKDTLMISRTPEVTC
VVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDG
SFFLYSRLTVDKSRWQEGNVFSCSVMHEALHNHYTQKSLSLSPGK*
hIgG4 Heavy Chain Constant Region
(SEQ ID NO: 20)
ASTKGPSVFPLAPCSRSTSESTAALGCLVKDYFPEPVTVSWNSGALT
SGVHTFPAVLQSSGLYSLSSVVTVPSSSLGTKTYTCNVDHKPSNTKV
DKRVESKYGPPCPSCPAPEFLGGPSVFLFPPKPKDTLMISRTPEVTC
VVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDG
SFFLYSRLTVDKSRWQEGNVFSCSVMHEALHNHYTQKSLSLSLGK*
hIgG4 mut Heavy Chain Constant Region
(SEQ ID NO: 14)
ASTKGPSVFPLAPCSRSTSESTAALGCLVKDYFPEPVTVSWNSGALT
SGVHTFPAVLQSSGLYSLSSVVTVPSSSLGTKTYTCNVDHKPSNTKV
DKRVESKYGPPCPPCPAPEFLGGPSVFLFPPKPKDTLMISRTPEVTC
VVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDG
SFFLYSRLTVDKSRWQEGNVFSCSVMHEALHNHYTQKSLSLSPGK*
hIgG4 mut Heavy Chain Constant Region
(SEQ ID NO: 21)
ASTKGPSVFPLAPCSRSTSESTAALGCLVKDYFPEPVTVSWNSGALT
SGVHTFPAVLQSSGLYSLSSVVTVPSSSLGTKTYTCNVDHKPSNTKV
DKRVESKYGPPCPPCPAPEFLGGPSVFLFPPKPKDTLMISRTPEVTC
VVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDG
SFFLYSRLTVDKSRWQEGNVFSCSVMHEALHNHYTQKSLSLSLGK*

In some instances, the heavy chain constant region in an anti-Galectin-9 antibody disclosed herein (e.g., G9.2-17) may have the C-terminal Lysine (K) residue removed for, e.g., manufacturing purposes. The corresponding amino acid sequences of those having no terminal K residue are provided below.

hIgG1 Heavy Chain Constant Region with No
C-Terminal Lysine
(SEQ ID NO: 24)
ASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSV
LTVLHQDWLNGKEYKCKVSNKALPAPIEKTISKAKGQPREPQVYTLP
PSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG*
hIgG1 LALA Heavy Chain Constant Region with No
C-Terminal Lysine
(SEQ ID NO: 25)
ASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKV
DKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSV
LTVLHQDWLNGKEYKCKVSNKALGAPIEKTISKAKGQPREPQVYTLP
PSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG*
hIgG4 Heavy Chain Constant Region with No
C-Terminal Lysine
(SEQ ID NO: 26)
ASTKGPSVFPLAPCSRSTSESTAALGCLVKDYFPEPVTVSWNSGALT
SGVHTFPAVLQSSGLYSLSSVVTVPSSSLGTKTYTCNVDHKPSNTKV
DKRVESKYGPPCPSCPAPEFLGGPSVFLFPPKPKDTLMISRTPEVTC
VVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDG
SFFLYSRLTVDKSRWQEGNVFSCSVMHEALHNHYTQKSLSLSPG*
hIgG4 Heavy Chain Constant Region with No
C-Terminal Lysine
(SEQ ID NO: 27)
ASTKGPSVFPLAPCSRSTSESTAALGCLVKDYFPEPVTVSWNSGALT
SGVHTFPAVLQSSGLYSLSSVVTVPSSSLGTKTYTCNVDHKPSNTKV
DKRVESKYGPPCPSCPAPEFLGGPSVFLFPPKPKDTLMISRTPEVTC
VVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDG
SFFLYSRLTVDKSRWQEGNVFSCSVMHEALHNHYTQKSLSLSLG*
hIgG4 mut Heavy Chain Constant Region with No
C-Terminal Lysine
(SEQ ID NO: 28)
ASTKGPSVFPLAPCSRSTSESTAALGCLVKDYFPEPVTVSWNSGALT
SGVHTFPAVLQSSGLYSLSSVVTVPSSSLGTKTYTCNVDHKPSNTKV
DKRVESKYGPPCPPCPAPEFLGGPSVFLFPPKPKDTLMISRTPEVTC
VVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDG
SFFLYSRLTVDKSRWQEGNVFSCSVMHEALHNHYTQKSLSLSPG*
hIgG4 mut Heavy Chain Constant Region with No
C-Terminal Lysine
(SEQ ID NO: 29)
ASTKGPSVFPLAPCSRSTSESTAALGCLVKDYFPEPVTVSWNSGALT
SGVHTFPAVLQSSGLYSLSSVVTVPSSSLGTKTYTCNVDHKPSNTKV
DKRVESKYGPPCPPCPAPEFLGGPSVFLFPPKPKDTLMISRTPEVTC
VVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDG
SFFLYSRLTVDKSRWQEGNVFSCSVMHEALHNHYTQKSLSLSLG*

In some embodiments, anti-Galectin-9 antibodies having any of the above heavy chain constant regions are paired with a light chain having the following light chain constant region:

Light Chain Constant Region
(SEQ ID NO: 11)
TVAAPSVFIFPPSDEQLKSGTASVVCLLNNFYPREAKVQWKVDNALQ
SGNSQESVTEQDSKDSTYSLSSTLTLSKADYEKHKVYACEVTHQGLS
SPVTKSFNRGEC

Exemplary full length anti-Galectin-9 antibodies are provided below:

G9.2-17 hIgG1 Heavy Chain (SEQ ID NO: 16)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PSSKSTSGGTAALGCLVKDYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK*
G9.2-17 hIgG1 Heavy Chain with No C-terminal Lysine Residue
(SEQ ID NO: 30)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PSSKSTSGGTAALGCLVKDYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPG*
G9.2-17 hIgG1 LALA Heavy Chain (SEQ ID NO: 17)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PSSKSTSGGTAALGCLVKDYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALGAPIE
KTISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK*
G9.2-17 hIgG1 LALA Heavy Chain with No C-terminal Lysine Residue
(SEQ ID NO: 31)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PSSKSTSGGTAALGCLVKDYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALGAPIE
KTISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPG*
G9.2-17 hIgG4 Heavy Chain (SEQ ID NO: 18)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSPGK*
G9.2-17 hIgG4 Heavy Chain with No C-terminal Lysine Residue
(SEQ ID NO: 32)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSPG*
G9.2-17 hIgG4 Heavy Chain (SEQ ID NO: 22)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK*
G9.2-17 hIgG4 Heavy Chain with No C-terminal Lysine Residue
(SEQ ID NO: 33)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLG*
G9.2-17 hIgG4 Fab Arm Exchange mut Heavy Chain (SEQ ID NO: 19)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSPGK*
G9.2-17 hIgG4 Fab Arm Exchange mut Heavy Chain with No C-terminal
Lysine Residue (SEQ ID NO: 34)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSPG*
G9.2-17 hIgG4 Fab Arm Exchange mut Heavy Chain (SEQ ID NO: 23)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK*
G9.2-17 hIgG4 Fab Arm Exchange mut Heavy Chain with No C-terminal
Lysine Residue (SEQ ID NO: 35)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLG*
Any of the above heavy chain can be paired with a Light Chain of
(SEQ ID NO: 15) shown below:
DIQMTQSPSSLSASVGDRVTITCRASQSVSSAVAWYQQKPGKAPKLLIYSASSLYSGVPSRFSGSRSG
TDFTLTISSLQPEDFATYYCQQSSTDPITFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC*

In some embodiments, the anti-Galectin-9 antibody comprises a heavy chain IgG1 constant region that has at least 80% (e.g., 80%, 85%, 90%, 91%, 92%, 93%, 94%, 95%, 96%, 97%, 98%, or 99% and any increment therein) sequence identity to SEQ ID NO: 10. In one embodiment, the constant region of the anti-Galectin-9 antibody comprises a heavy chain IgG4 constant region comprising SEQ ID NO: 10. In one embodiment, the constant region of the anti-Galectin-9 antibody comprises a heavy chain IgG1 constant region consisting of SEQ ID NO: 10.

In some embodiments, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 20. In one embodiment, the constant region of the anti-Galectin-9 antibody comprises a heavy chain IgG4 constant region comprising SEQ ID NO: 20. In one embodiment, the constant region of the anti-Galectin-9 antibody comprises a heavy chain IgG4 constant region consisting of SEQ ID NO: 20.

In some embodiments, the constant region is from human IgG4. In one embodiment, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 13. In one embodiment, the anti-Galectin-9 antibody comprises a heavy chain IgG4 constant region comprising SEQ ID NO: 13. In one embodiment, the anti-Galectin-9 antibody comprises a heavy chain IgG4 constant region consisting of SEQ ID NO: 13.

In some embodiments, the constant region is from human IgG4. In one embodiment, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 20. In one embodiment, the anti-Galectin-9 antibody comprises a heavy chain IgG4 constant region comprising SEQ ID NO: 20. In one embodiment, the anti-Galectin-9 antibody comprises a heavy chain IgG4 constant region consisting of SEQ ID NO: 20.

In any of these embodiments, the anti-Galectin-9 antibody comprises a light chain constant region that has at least 80% (e.g., 80%, 85%, 90%, 91%, 92%, 93%, 94%, 95%, 96%, 97%, 98%, or 99% and any increment therein) sequence identity to SEQ ID NO: 11. In some embodiments, the anti-Galectin-9 antibody comprises alight chain constant region comprising SEQ ID NO: 11. In some embodiments, the anti-Galectin-9 antibody comprises a light chain constant region consisting of SEQ ID NO: 11.

In some embodiments, the IgG is a mutant with minimal Fc receptor engagement. In one example, the constant region is from a human IgG1 LALA. In one embodiment, the anti-Galectin-9 antibody comprises a heavy chain IgG1 constant region that has at least 80% (e.g., 80%, 85%, 90%, 91%, 92%, 93%, 94%, 95%, 96%, 97%, 98%, or 99% and any increment therein) sequence identity to SEQ ID NO: 12. In one embodiment, the anti-Galectin-9 antibody comprises a heavy chain IgG1 constant region comprising SEQ ID NO: 12. In one embodiment, the anti-Galectin-9 antibody comprises a heavy chain IgG1 constant region consisting of SEQ ID NO: 12.

In some embodiments, the anti-Galectin-9 antibody comprises a modified constant region. In some embodiments, the anti-Galectin-9 antibody comprise a modified constant region that is immunologically inert, e.g., does not trigger complement mediated lysis, or does not stimulate antibody-dependent cell mediated cytotoxicity (ADCC). ADCC activity can be assessed using methods disclosed in U.S. Pat. No. 5,500,362. In other embodiments, the constant region is modified as described in Eur. J. Immunol. (1999) 29:2613-2624; WO/1999/058572; and/or UK Patent Application No. 9809951.8. In some embodiments, the IgG4 constant region is a mutant with reduced heavy chain exchange. In some embodiments, the constant region is from a human IgG4 Fab Arm Exchange mutant S228P.

In one embodiment, the constant region of the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 14. In one embodiment, the constant region of the anti-Galectin-9 antibody comprises a heavy chain IgG4 constant region comprising SEQ ID NO: 14. In one embodiment, the constant region of the anti-Galectin-9 antibody comprises a heavy chain IgG4 constant region consisting of SEQ ID NO: 14.

In one embodiment, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 21. In one embodiment, the anti-Galectin-9 antibody comprises a heavy chain IgG4 constant region comprising SEQ ID NO: 21. In one embodiment, the anti-Galectin-9 antibody comprises a heavy chain IgG4 constant region consisting of SEQ ID NO: 21.

In some embodiments, the anti-Galectin-9 antibody has chains corresponding to SEQ ID NO: 15 for the light chains; and the amino acid sequences of exemplary heavy chains correspond to SEQ ID NO: 10 (hIgG1); 12 (hIgG1 LALA); 13 (hIgG4); 20 (hIgG4); 14 (hIgG4 mut); and 21 (hIgG4 mut).

In some embodiments, the anti-Galectin-9 antibody has a light chain comprising, consisting essentially of, or consisting of SEQ ID NO: 15. In some embodiments, the anti-Galectin-9 antibody has a heavy chain comprising, consisting essentially of, or consisting of any one of the sequences selected from the group consisting of SEQ ID NO: 16-19, 22 and 23. In some embodiments, the anti-Galectin-9 antibody has a light chain comprising, consisting essentially of, or consisting of SEQ ID NO: 15 and a heavy chain comprising, consisting essentially of, or consisting of any one of the sequences selected from the group consisting of SEQ ID NO: 16-19. In some embodiments, the anti-Galectin-9 antibody has a light chain comprising SEQ ID NO: 15 and a heavy chain comprising any one of the sequences selected from the group consisting of SEQ ID NO: 16-19, 22 and 23. In some embodiments, the anti-Galectin-9 antibody has a light chain consisting essentially of SEQ ID NO: 15 and a heavy chain consisting essentially of any one of the sequences selected from the group consisting of SEQ ID NO: 16-19, 22 and 23. In some embodiments, the anti-Galectin-9 antibody has a light chain consisting of SEQ ID NO: 15 and a heavy chain consisting of any one of the sequences selected from the group consisting of SEQ ID NO: 16-19, 22 and 23. In one specific embodiment, the anti-Galectin-9 antibody has a light chain consisting essentially of SEQ ID NO: 15 and a heavy chain consisting essentially of SEQ ID NO: 19. In another specific embodiment, the anti-Galectin-9 antibody has a light chain consisting essentially of SEQ ID NO: 15 and a heavy chain consisting essentially of SEQ ID NO: 20.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 16. In one embodiment, the anti-Galectin-9 antibody comprises a heavy chain sequence comprising SEQ ID NO: 16. In one embodiment, the anti-Galectin-9 antibody comprises a heavy chain sequence consisting of SEQ ID NO: 16.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 17. In one embodiment, the anti-Galectin-9 antibody comprises a heavy chain sequence comprising SEQ ID NO: 17. In one embodiment, the anti-Galectin-9 antibody comprises a heavy chain sequence consisting of SEQ ID NO: 17.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 18. In one embodiment, the anti-Galectin-9 antibody comprises a heavy chain sequence comprising SEQ ID NO: 18. In one embodiment, the anti-Galectin-9 antibody comprises a heavy chain sequence consisting of SEQ ID NO: 18.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 22. In one embodiment, the anti-Galectin-9 antibody comprises a heavy chain sequence comprising SEQ ID NO: 22. In one embodiment, the anti-Galectin-9 antibody comprises a heavy chain sequence consisting of SEQ ID NO: 22.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 19. In one embodiment, the anti-Galectin-9 antibody comprises a heavy chain sequence comprising SEQ ID NO: 19. In one embodiment, the anti-Galectin-9 antibody comprises a heavy chain sequence consisting of SEQ ID NO: 19.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 23. In one embodiment, the anti-Galectin-9 antibody comprises a heavy chain sequence comprising SEQ ID NO: 23. In one embodiment, the anti-Galectin-9 antibody comprises a heavy chain sequence consisting of SEQ ID NO: 23.

In any of these embodiments, the anti-Galectin-9 antibody comprises a light chain sequence having at least 80% (e.g., 80%, 85%, 90%, 91%, 92%, 93%, 94%, 95%, 96%, 97%, 98%, or 99% and any increment therein) sequence identity to SEQ ID NO: 15. In some embodiments, the anti-Galectin-9 antibody comprises alight chain sequence comprising SEQ ID NO: 15. In some embodiments, the anti-Galectin-9 antibody comprises a light chain sequence consisting of SEQ ID NO: 15.

In specific examples, the anti-Galectin-9 antibody used in the treatment methods disclosed herein has a heavy chain of SEQ ID NO:19 and a light chain of SEQ ID NO:15. In some embodiments, the anti-Galectin-9 antibody used in the treatment methods disclosed herein is G9.2-17 IgG4. In some examples, such an anti-Galectin-9 antibody does not have the C-terminal lysine residue in its heavy chain.

### II. Preparation of Anti-Galectin-9 Antibodies

Antibodies capable of binding Galectin-9 as described herein can be made by any method known in the art, including but not limited to, recombinant technology. One example is provided below.

Nucleic acids encoding the heavy and light chain of an anti-Galectin-9 antibody as described herein can be cloned into one expression vector, each nucleotide sequence being in operable linkage to a suitable promoter. In one example, each of the nucleotide sequences encoding the heavy chain and light chain is in operable linkage to a distinct promoter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementarity ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters (M. Brown et al., Cell, 49:603-612 (1987); Gossen and Bujard (1992); M. Gossen et al., Natl. Acad. Sci. USA, 89:5547-5551 (1992)) combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of poly adenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-Galectin-9 antibody, as also described herein. The recombinant expression vector can be introduced into a suitable host cell (e.g., a dhfr− CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-Galectin-9 antibody and the other encoding the light chain of the anti-Galectin-9 antibody. Both of the two recombinant expression vectors can be introduced into a suitable host cell (e.g., dhfr− CHO cell) by a conventional method, e.g., calcium phosphate-mediated transfection. Alternatively, each of the expression vectors can be introduced into a suitable host cell. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-Galectin-9 antibody as described herein, vectors (e.g., expression vectors) containing such; and host cells comprising the vectors are within the scope of the present disclosure.

Anti-Galectin-9 antibodies thus prepared can be characterized using methods known in the art, whereby reduction, amelioration, or neutralization of Galectin-9 biological activity is detected and/or measured. For example, in some embodiments, an ELISA-type assay is suitable for qualitative or quantitative measurement of Galectin-9 inhibition of Dectin-1 or TIM-3 signaling.

The bioactivity of an anti-Galectin-9 antibody can verified by incubating a candidate antibody with Dectin-1 and Galectin-9, and monitoring any one or more of the following characteristics: (a) binding between Dectin-1 and Galectin-9 and inhibition of the signaling transduction mediated by the binding; (b) preventing, ameliorating, or treating any aspect of a hematologic malignancy; (c) blocking or decreasing Dectin-1 activation; (d) inhibiting (reducing) synthesis, production or release of Galectin-9. Alternatively, TIM-3 can be used to verify the bioactivity of an anti-Galectin-9 antibody using the protocol described above. Alternatively, CD206 can be used to verify the bioactivity of an anti-Galectin-9 antibody using the protocol described above.

In some embodiments, bioactivity or efficacy is assessed in a subject, e.g., by measuring peripheral and intra-tumoral T cell ratios, T cell activation, or by macrophage phenotyping.

Additional assays to determine bioactivity of an anti-Galectin-9 antibody include measurement of CD8+ and CD4+ (conventional) T-cell activation (in an in vitro or in vivo assay, e.g., by measuring inflammatory cytokine levels, e.g., IFNgamma, TNFalpha, CD44, ICOS granzymeB, Perform, IL2 (upregulation); CD26L and IL-10 (downregulation)); measurement of reprogramming of macrophages (in vitro or in vivo), e.g., from the M2 to the M1 phenotype (e.g., increased MHCII, reduced CD206, increased TNF-alpha and iNOS), Alternatively, levels of ADCC can be assessed, e.g., in an in vitro assay, as described herein.

### III. Methods of Treatment

The present disclosure provides methods for treating a hematologic malignancy, which are cancers that begin in blood-forming tissue, such as the bone marrow or in the cells of the immune system. Hematological malignancies include acute lymphoblastic leukemia, chronic lymphocytic leukemia, lymphomas, multiple myeloma, acute myelogenous leukemia, chronic myelogenous leukemia, myelodysplastic syndromes and the myeloproliferative neoplasms, such as essential thrombocythemia, polycythemia vera and myelofibrosis. A subject having a hematological malignancy can be identified by routine medical examination, e.g., laboratory tests, organ functional tests, CT scans, or ultrasounds.

### A. Pharmaceutical Compositions

Any of the anti-Gal9 antibodies disclosed herein (e.g., G9.2-17 (IgG4)), as well as the encoding nucleic acids or nucleic acid sets, vectors comprising such, can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. The antibodies, or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

In some examples, the pharmaceutical composition is an injectable formulation, Injectable formulations may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous infusion, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

### (B) Treatment of Hematological Malignancies

In some embodiments, the present disclosure provides a method for treating a hematologic malignancy, for example, a hematologic malignancy involving Gal9+ cancer cells using an effective amount of an anti-Galectin-9 antibody described herein, including but not limited to, G9.2-17 IgG4 (having a heavy chain of SEQ ID NO:19 and a light chain of SEQ ID NO:15).

### Exemplary Target Hematological Malignancies

In some examples, the method disclosed herein is applied to a human patient having a leukemia, for example, acute myelogenous leukemia (AML) or acute lymphoblastic leukemia (ALL), including T-cell ALL and B-cell ALL. In other examples, the method disclosed herein is applied to a human patient having a lymphoma, e.g., those disclosed herein. In yet another example, the method disclosed herein is applied to a human patient having myelodysplastic syndromes (MDS).

Acute myeloid leukemia (AML) is a blood cancer that starts in the blood and bone marrow and can progress rapidly. AML involves abnormal white blood cells produced by bone marrow, which fill the blood and bone marrow, leaving less space for healthy cells. Symptoms associated with AML include bleeding and anemia. Left untreated, AML can be life threatening.

Acute lymphoblastic leukemia (acute lymphocytic leukemia or ALL) is a cancer of the bone marrow that can spread quickly to blood cells. ALL causes bone marrow to make too many immature white blood cells (lymphoblasts). These abnormal cells crowd out healthy red and white blood cells and platelets in the blood and bone marrow, making it difficult for the body to fight infection and disease. As the most common childhood leukemia and cancer, ALL typically occurs before age 15. T-cell acute lymphoblastic leukemia (T-ALL) is an aggressive malignant neoplasm of the bone marrow and account for about 20% of all ALL cases. B-cell acute lymphoblastic leukemia (B-ALL) is the most common type of cancer in children, usually affecting B cells in a patient.

Myelodysplastic syndromes (MDS) are conditions that can occur when the blood-forming cells in the bone marrow become abnormal. This leads to low numbers of one or more types of blood cells. In MDS, some of the cells in the bone marrow are abnormal (dysplastic) and have problems making new blood cells. Many of the blood cells formed by these bone marrow cells are defective. Defective cells often die earlier than normal cells, and the body also destroys some abnormal blood cells, leaving the patient without enough normal blood cells. Different cell types can be affected, although the most common finding in MDS is a shortage of red blood cells (anemia).

In some instances, the subject may be a human patient having a refractory disease, for example, a refractory AML, a refractory ALL, or a refractory MDS. As used herein, “refractory” refers to the tumor that does not respond to or becomes resistant to a treatment. In some instances, the subject may be a human patient having a relapsed disease, for example, a relapsed AML, a relapsed ALL, a relapsed MDS, or a relapsed cholangiocarcinoma. As used herein, “relapsed” or “relapses” refers to the tumor that returns or progresses following a period of improvement (e.g., a partial or complete response) with treatment.

In some instances, the subject is a human patient having an elevated level of Galectin-9 as relative to a control level. The level of Galectin-9 can be a plasma or serum level of Galectin-9 in the human patient. In other examples, the level of Galectin-9 is the level of Galectin-9 of cancer cells within the tumor. In other examples, the level of Galectin-9 is the level of Galectin-9 of immune cells within the tumor. In other examples, the level of Galectin-9 can be the level of cell-surface Galectin-9, for example the level of Galectin-9 on cancer cells. In one example, the level of Galectin-9 can be the level of Galectin-9 expressed cancer cells, e.g., on the surface of cancer cells, or Galectin-9 expressed in immune cells. In some examples, the control level represents the level of Galectin-9 in healthy subjects. In some embodiments, the control level may be a baseline level prior to treatment.

To identify such a subject, a suitable biological sample can be obtained from a subject who is suspected of having the hematologic malignancy and the biological sample can be analyzed to determine the level of Galectin-9 contained therein (e.g., free, cell-surface expressed, or total) using conventional methods, e.g., ELISA or FACS. In some embodiments, organoid cultures are prepared, e.g., as described herein, and used to assess Galectin-9 levels in a subject. Single cells derived from certain fractions obtained as part of the organoid preparation process are also suitable for assessment of Galectin-9 levels in a subject. In some instances, an assay for measuring the level of Galectin-9, either in free form or expressed on cell surface, involves the use of an antibody that specifically binds the Galectin-9 (e.g., specifically binds human Galectin-9). Any of the anti-Galectin-9 antibodies known in the art can be tested for suitability in any of the assays described above and then used in such assays in a routine manner. In some embodiments, an antibody described herein (e.g., a G9.2-17 antibody) can be used in such as assay. In some embodiments, an antibody described in U.S. Pat. No. 10,344,091 and WO2019/084553, the relevant disclosures of each of which are incorporated by reference for the purpose and subject matter referenced herein. In some examples, the anti-Galectin-9 antibody is a Fab molecule. Assay methods for determining Galectin-9 levels as disclosed herein are also within the scope of the present disclosure.

In some embodiments, a human patient to be treated by any of the methods disclosed herein may meet one or more of the inclusion and exclusion criteria listed in Example 1 below. For example, such a human patient may meet all of the inclusion and exclusion criteria listed in Example 1 below.

### Exemplary Treatment Conditions

In some embodiments, a pharmaceutical composition comprising an effective amount of the anti-Gal9 antibody disclosed herein (e.g., G9.2-17 IgG4) may be given to a subject (e.g., a human patient, who can be an adult or a child) in need of the treatment, e.g., at a suitable dosage and a suitable dosing frequency. For example, the antibody may be administered to the subject via a suitable route (e.g., intravenous infusion) at a dose of about 2 mg/kg (e.g., about 4 mg/kg) to about 32 mg/kg once every week to once every 6 weeks. See also disclosures herein.

The term “about” or “approximately” means within an acceptable error range for the particular value as determined by one of ordinary skill in the art, which are dependent in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

An effective amount of the pharmaceutical composition described herein can be administered to a subject (e.g., a human) in need of the treatment via a suitable route, systemically or locally. In some embodiments, the anti-Galectin-9 antibodies are administered by intravenous administration, e.g., as a bolus or by continuous infusion over a period of time, by intramuscular, intraperitoneal, intracerebrospinal, subcutaneous, intra-arterial, intra-articular, intrasynovial, intrathecal, intratumoral, sub-urothelial, oral, inhalation or topical routes. In one embodiment, the anti-Galectin-9 antibody is administered to the subject by intravenous infusion. In one embodiment, the anti-galectin-9 antibody is administered to the subject intraperitoneally.

As used herein, “an effective amount” refers to the amount of each active agent required to confer therapeutic effect on the subject, either alone or in combination with one or more other active agents. In some embodiments, the therapeutic effect is reduced Galectin-9 activity and/or amount/expression, reduced Dectin-1 signaling, reduced TIM-3 signaling, reduced CD206 signaling, and/or increased anti-tumor immune responses in the tumor microenvironment. Non-limiting examples of increased anti-tumor responses include increased activation levels of effector T cells or switching of the TAMs from the M2 to the M1 phenotype. In some cases, the anti-tumor response includes increased ADCC responses. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner.

Empirical considerations, such as the half-life, generally contribute to the determination of the dosage. For example, antibodies that are compatible with the human immune system, such as humanized antibodies or fully human antibodies, are in some instances used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, a symptom of the disease or disorder, or the predisposition toward the disease or disorder. Non-limiting examples of treatment include reducing tumor size, delaying tumor growth, alleviating lesions and/or delaying development of lesions, and/or elongating survival of human patients having the target hematological malignancies such as AML, MDS, or ALL (T cell or B cell ALL), which may be refractory and/or relapsed.

Alleviating a target disease/disorder includes delaying the development or progression of the disease or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

In one example, dosages for an antibody as described herein are determined empirically in individuals who have been given one or more administration(s) of the antibody. Individuals are given incremental dosages of the antibody antagonist. To assess efficacy of the antibody antagonist, an indicator of the disease/disorder, such as any of the disclosed indicators, can be followed.

### Monotherapy

In some embodiments, any of the anti-Gal9 antibodies disclosed herein, such as G9.2-17(IgG4), may be used as a monotherapy agent (the only active agent) for treating the target hematological malignancy, such as AML, MDS, or ALL. Given the direct cell killing effect of the antibody against Gal9+ cancer cells, the anti-Gal9 antibody disclosed herein is expected to be effective in treating hematological cancer involving Gal9+ cancer cells as a single therapeutic agent.

In some embodiments, the anti-Gal9 antibody can be administered to a patient once every week to once every six weeks. e.g., via intravenous infusion. In some examples, the antibody may be administered once every 1-4 weeks, for example, every 2-4 weeks. In one example, the antibody may be administered once every week. In another example, the antibody may be administered every 2 weeks.

In some embodiments, the anti-Galectin 9 antibody disclosed herein (e.g., G9.2-17 IgG4) is administered via a 30-minute to 6-hour infusion period intravenously. In some examples the intravenous infusion of the anti-Galectin 9 antibody may be performed for 30 minutes to 2 hours. In other examples, the anti-Galectin 9 antibody may be administered via a long infusion period, for example, about 2-6 hours, e.g., about 2-4 hours or about 4-6 hours. In specific examples, examples anti-Galectin 9 antibody may be infused intravenous in a period of about 3 hours, about 4 hours, about 5 hours, or about 6 hours.

In some embodiments, the anti-Galectin-9 antibody disclosed herein (e.g., G9.2-17(IgG4)) for use in treating a hematological malignancy (e.g., those disclosed herein such as AML, MDS, or ALL) can be administered to the subject at a dose of about 0.2 mg/kg to about 32 mg/kg, e.g., the dose may be selected from 0.2 mg/kg, 0.63 mg/kg, 2 mg/kg, 4 mg/kg, 6 mg/kg, 6.3 mg/kg, 7.5 mg/kg, 8 mg/kg, 10 mg/kg, 12 mg/kg, 16 mg/kg, 32 mg/kg, or a higher dose level. In some embodiments, the anti-Galectin-9 antibody may be administered to the subject at a dose of about 1 mg/kg to about 32 mg/kg, e.g., the dose may be selected from 2 mg/kg, 4 mg/kg, 8 mg/kg, 12 mg/kg, 16 mg/kg, 32 mg/kg or a higher dose level. In some examples, the anti-Galectin-9 antibody may be administered to the subject at a dose of about 0.2 mg/kg to about 32 mg/kg, e.g., the dose may be selected from 0.2 mg/kg, 0.63 mg/kg, 2 mg/kg, 4 mg/kg, 6 mg/kg, 6.3 mg/kg, 7.5 mg/kg, 10 mg/kg, 12 mg/kg, 16 mg/kg, 32 mg/kg or a higher dose level.

In one example, the anti-Galectin-9 antibody disclosed herein (e.g., G9.2-17(IgG4)) is administered to the subject at a dose of 2 mg/kg every week. In another example, the anti-Galectin-9 antibody disclosed herein (e.g., G9.2-17(IgG4)) is administered to the subject at a dose of 4 mg/kg every week. In yet another example, the anti-Galectin-9 antibody disclosed herein (e.g., G9.2-17(IgG4)) is administered to the subject at a dose of 6.3 mg/kg every week. In yet another example, the anti-Galectin-9 antibody disclosed herein (e.g., G9.2-17(IgG4)) is administered to the subject at a dose of 7.5 mg/kg every week. In still another example, the anti-Galectin-9 antibody disclosed herein (e.g., G9.2-17(IgG4)) is administered to the subject at a dose of 10 mg/kg every week. In yet another example, the anti-Galectin-9 antibody disclosed herein (e.g., G9.2-17(IgG4)) is administered to the subject at a dose of 12 mg/kg every week. In another example, the anti-Galectin-9 antibody disclosed herein (e.g., G9.2-17(IgG4)) is administered to the subject at a dose of 16 mg/kg every week.

In one example, the anti-Gal9 antibody disclosed herein (e.g., G9.2-17(IgG4)) can be given to a human patient having a target hematological malignancy such as AML, MDS, or ALL at 4 mg/kg (e.g., by i.v.) once every week or once every two weeks. In another example, the anti-Gal9 antibody can be given to the human patient at 7.5 mg/kg (e.g., by i.v.) once every week or once every two weeks. In yet another example, the anti-Gal9 antibody can be given to the human patient at 12 mg/kg (e.g., by i.v.) once every week or once every two weeks. In still another example, the anti-Gal9 antibody can be given to the human patient at 16 mg/kg (e.g., by i.v.) once every week or once every two weeks.

In some instances, a flat dosing schedule may be adopted in any of the treatment methods disclosed herein. For example, the anti-Gal-9 antibody such as G9.2-17(IgG4) disclosed herein may be administered to a subject at a flat dose, e.g., about 410 mg to about 1120 mg, once every week to once every 4 weeks (e.g., once every week or once every two 5 weeks). In some examples, the anti-Gal-9 antibody is administered to a subject at a about 410 mg to about 505 mg (e.g., about 410 mg to about 450 mg) once every week. In some examples, the anti-Gal-9 antibody is administered to a subject at a about 410 mg to about 505 mg (e.g., about 410 to about 450 mg) once every two weeks. In some examples, the anti-Gal-9 antibody is administered to a subject at a about 650 mg to about 800 mg (e.g., about 650 mg to about 700 mg) once every week. In some examples, the anti-Gal-9 antibody is administered to a subject at a about 650 mg to about 800 mg (e.g., about 650 mg to about 700 mg) once every two weeks. In some examples, the anti-Gal-9 antibody is administered to a subject at a about 1040 mg to about 1,280 mg (e.g., about 1040 to about 1120 mg) once every week. In some examples, the anti-Gal-9 antibody is administered to a subject at a about 1040 mg to about 1,280 mg (e.g., about 1040 to about 1120 mg) once every two weeks. In some examples, the anti-Gal-9 antibody is administered to a subject at a about 2080 mg to about 2560 mg (e.g., about 2080 to about 2400 mg) once every week. In some examples, the anti-Gal-9 antibody is administered to a subject at a about 2080 mg to about 2560 mg (e.g., about 2080 to about 2400 mg) once every two weeks.

In other instances, the flat dosing schedule may comprise about 130 mg to about 320 mg once every week to once every six weeks (e.g., every 1 week, every 2 weeks, every 3 weeks, every 4 weeks, every 5 weeks, or every 6 weeks). For example, the flat dosing schedule may comprise about 130 mg to about 160 mg once every week. Alternatively, the flat dosing schedule may comprise about 130 mg to about 160 mg once every two weeks. In another example, the flat dosing schedule may comprise about 260 mg to about 320 mg once every week. Alternatively, the flat dosing schedule may comprise about 260 mg to about 320 mg once every two weeks.

In some instances, the treatment period may be 6 months to 12 months. In other instances, the treatment period may be 12 months to 24 months. In other instances, the treatment period may be longer than 24 months.

In some embodiments, the dosage(s) is adjusted in accordance with the patient's response to treatment. In some embodiments, the dosages are altered between treatment intervals. In some embodiments, the treatment may be temporarily stopped. In some embodiments, the treatment may be temporarily stopped. In some embodiments, anti-Galectin-9 therapy is temporarily stopped. In some embodiments, a checkpoint inhibitor therapy employed in combination with the anti-Galectin-9 antibody is temporarily stopped. In some embodiments, both are temporarily stopped.

In some instances, a human patient may start with a low dose of the anti-Galectin-9 antibody such as G9.2-17 (IgG4) disclosed herein, for example, 4 mg/kg, 6.3 mg/kg or 7.5 mg/kg. The dose may be elevated to, e.g., 10 mg/kg, 12 mg/kg, 16 mg/kg, or 32 mg/kg when applicable, which can be determined by a medical practioner. Similarly, a human patient may start with a long dosing frequency (e.g., every two weeks) and adjust to a short dosing frequence (e.g., every week), or vice versa, when applicable.

### Combined Therapy

In some embodiments, any of the anti-Gal9 antibodies described herein (e.g., a G9.2-17 antibody such as G9.2-17(IgG4) as disclosed herein) can be used in combination with a second therapeutic, e.g., a chemotherapeutic agent, for treating a target hematological malignancy as disclosed herein (e.g., AML, MDS, or ALL). Selection of a suitable chemotherapeutic agent or other combinatorial agent(s) and treatment modalities would depend on various factors, including the target hematologic malignancy, severity of the disease, age, gender, treatment history, resistant to prior treatment, etc., which are within the knowledge of a medical practioner.

For example, the anti-Gal9 antibody such as G9.2-17(IgG4) can be used in combination with a suitable chemotherapeutic agent for treating AML. Exemplary chemotherapeutic agents include, but are not limited to, Azacitidine, Cerubidine (Daunorubicin Hydrochloride), Cyclophosphamide, Cytarabine, Daunorubicin Hydrochloride and Cytarabine Liposome, Daurismo (Glasdegib Maleate), Dexamethasone, Doxorubicin Hydrochloride, Enasidenib Mesylate, Gemtuzumab Ozogamicin, Gilteritinib Fumarate, Glasdegib Maleate, Idamycin PFS (Idarubicin Hydrochloride), Idarubicin Hydrochloride, Idhifa (Enasidenib Mesylate), Ivosidenib, Midostaurin, Mitoxantrone Hydrochloride, Mylotarg (Gemtuzumab Ozogamicin), Onureg (Azacitidine), Prednisone, Rubidomycin (Daunorubicin Hydrochloride), Rydapt (Midostaurin), Tabloid (Thioguanine), Thioguanine, Tibsovo (Ivosidenib), Trisenox (Arsenic Trioxide), Venclexta (Venetoclax), Venetoclax, Vincristine Sulfate, Vyxeos (Daunorubicin Hydrochloride and Cytarabine Liposome), Xospata (Gilteritinib Fumarate), Sabatolimab, or a combination thereof. In some instances, the second therapeutic agent for co-use with the anti-Gal9 antibody for treating AML can be an PD-1 inhibitor. Examples include, but are not limited to, anti-PD-1 antibodyes (e.g., pembrolizumab, nivolumab, tislelizumab, dostarlimab, and cemiplimab) or anti-PD-L1 antibodies (e.g., durvalumab, avelumab, and atezolizumab).

In other examples, the anti-Gal9 antibody such as G9.2-17(IgG4) can be used in combination with a suitable chemotherapeutic agent for treating MDS. Exemplary chemotherapeutic agents include, but are not limited to, Azacitidine (e.g., for both low- and high-risk patients with all sub-types of MDS), Decitabine (e.g., for both low- and high-risk patients with all sub-types of MDS), Lenalidomide (e.g., for transfusion-dependent MDS patients with isolated del(5q) and with a low or intermediate-1 risk IPSS score), Luspatercept-aamt (e.g., for adult MDS patients with ring sideroblasts (MDS-RS) or myelodysplastic/myeloproliferative neoplasms with ring sideroblasts and thrombocytosis (MDS/MPN-RS-T), who, in some instances, have been failed an erythropoiesis stimulating agent (ESA) or are unlikely to respond to an ESA, and are requiring transfusions of 2 or more red blood cell (RBC) units over 8 weeks); ASTX727 (e.g., for adult patients with MDS or CMML who are intermediate-1, intermediate-2, and high-risk IPSS groups including previously treated and untreated, de novo and secondary MDS including patients with refractory anemia, refractory anemia with ringed sideroblasts, refractory anemia with excess blasts and CMML); Luspatercept, Decitabine/Cedazuridine, Pevonedistat, Magrolimab, or a combination thereof.

In some embodiments, the anti-Gal9 antibody can be administered concurrently with any of the second therapeutic agent as disclosed herein. In some embodiments, the anti-Gal9 antibody can be administered before the second therapeutic agent. Alternatively, the anti-Gal9 antibody can be administered after the second therapeutic agent. In some instances, the second therapeutic agent is administered systemically. In other instances, the second therapeutic agent is administered locally. In some examples, the second therapeutic agent is administered by intravenous administration, e.g., as a bolus or by continuous infusion over a period of time, by intramuscular, intraperitoneal, intracerebrospinal, subcutaneous, intra-arterial, intra-articular, intravesical, intrasynovial, intrathecal, intratumoral, or sub-urothelial route. In one embodiment, the second therapeutic agent is administered to the subject by intravenous infusion.

### Monitoring Treatment Responses

Treatment efficacy for a target hematologic malignancy as disclosed herein can be assessed by methods well-known in the art.

In some embodiments, anti-tumor activity of any of treatment methods disclosed herein may be monitored by conventional approaches. For example, before, during, and after the administration of the antibody and optionally the second therapeutic agent, cancerous cells and/or biomarkers in a subject can be measured in a biological sample, such as blood, serum, plasma, urine, peritoneal fluid, and/or a biopsy from a tissue or organ. Results thus obtained can be analyzed to assess treatment efficacy. For examples, the levels of Gal9+ cancer cells may be measured before, during, and after the treatment to assess the direct killing effect of the anti-Gal9 antibody such as G9.2-17(IgG4).

Alternatively or in addition, response to treatment can also be characterized by one or more of immunophenotype (e.g., to assess immune modulating effects) in blood and tumors, cytokine profile (serum), soluble galectin-9 levels in blood (serum or plasma), galectin-9 tumor tissue expression levels and pattern of expression by immunohistochemistry (tumor, stroma, immune cells), tumor mutational burden (TMB), mismatch repair status, or tumor markers relevant for the disease (e.g., as measured at approximately 3 months, 6 months or 12 months, or at a later time or at any other clinically indicated time point).

In some examples, changes in levels of immune cells and immune cell markers in the blood or in tumors, e.g., immune activation, may be determined before and after the treatment. Such changes can be measured in patient blood and tissue samples using methods known in the art, such as multiplex flow cytometry and multiplex immunohistochemistry. For example, a panel of phenotypic and functional PBMC immune markers can be assessed at baseline prior to commencement of the treatment and at various time point during treatment. Table 2 lists non-limiting examples of markers useful for these assessment methods. Flow cytometry (FC) is a fast and highly informative method of choice technology to analyze cellular phenotype and function and has gained prominence in immune phenotype monitoring. It allows for the characterization of many subsets of cells, including rare subsets, in a complex mixture such as blood, and represents a rapid method to obtain large amounts of data. Advantages of FC are high speed, sensitivity, and specificity. Standardized antibody panels and procedures can be used to analyze and classify immune cell subtypes. Multiplex IHC is a powerful investigative tool, which provides objective quantitative data describing the tumor immune context in both immune subset number and location and allows for multiple markers to be assessed on a single tissue section. Computer algorithms can be used to quantify IHC-based biomarker content from whole slide images of patient biopsies, combining chromogenic IHC methods and stains with digital pathology approaches.

TABLE 2
PBMC phenotyping markers

| | PBMC phenotyping markers | | PBMC phenotyping markers |
|---|---|---|---|
| CD3 | Total T cells | CD16 | NK cells |
| CD4 | CD4+ T cells | CD11b | Monocytes/macrophages |
| CD8 | CD8+ T cells | CD11c | Monocytes/macrophages, DCs |
| CD25 | Treg activation | CD14 | Monocyte subsets, |
| | | | macrophages |
| CD27 | T cell maturation; B cell | CD33 | Total myeloid cells |
| | naïve/memory | | |
| CD38 | T cell maturation; B cell | FceR1 a | Antigen presenting DC cells |
| | naïve/memory | | |
| CD45RA | Naïve/memory cells | CD19 | Total B cells |
| CD45RO | Naïve/memory cells | T-bet | T cells subsets |
| CD56 | NKT/NK cells (T cell subset) | gdTCR | Gamma delta T cells |
| CD127 | T cell subsets | CD274 | Checkpoint |
| | | (PDL-1) | |
| CD152 | Checkpoint | Tim-3 | Checkpoint |
| (CLTA-4) | | | |
| CD279 | Checkpoint | TCRVa24- | iNKT cells |
| (PD-1) | | Ja18 | |
| FoxP3 | Treg cells | Live/dead | General |
| HLA-DR | Activation/Antigen presentation | CD45 | General |

A subject being treated by any of the anti-Gal9 antibodies disclosed herein (e.g., G9.2-17(IgG4)), either alone or in combination with a second therapeutic agent (e.g., a chemotherapeutic agent) as disclosed herein, may be monitored for occurrence of adverse effects (for example, severe adverse effects). Exemplary adverse effects to monitor are provided in Example 1 below. If occurrence of adverse effects is observed, treatment conditions may be changed for that subject. For example, the dose of the anti-galectin-9 antibody may be reduced and/or the dosing interval may be extended. Suitability and extent of reduction may be assessed by a qualified clinician. In some embodiments, one or more dose reductions by about 10% to about 80% of a previous dose level can be implemented. In some embodiments, one or more dose reductions by about 10% to about 20%, about 20% to about 30%, about 30% to about 40%, about 40% to about 50%, about 50% to about 60%, or about 70% to about 80% of a previous dose level can be implemented. In some embodiments, one or more dose reductions by 10% to 20%, 20% to 30%, 30% to 40%, 40% to 50%, 50% to 60%, or 70% to 80% of a previous dose level can be implemented. In some embodiments, one or more dose reductions by about 10%, by about 20%, by about 30%, by about 40%, by about 50%, by about 60%, by about 70%, or by about 80% of a previous dose level can be implemented. In some embodiments, one or more dose reductions by 10%, by 20%, by 30%, by 40%, by 50%, by 60%, by 70%, or by 80% of a previous dose level are implemented. Alternatively, or in addition, the dose of the second therapeutic agent can be reduced and/or the dosing interval of the checkpoint inhibitor may be extended. In some instances (e.g., occurring of life-threatening adverse effects), the treatment may be terminated.

Any of the methods disclosed herein may involve reducing the dose of the anti-Gal9 antibody when one or more adverse events (e.g., those disclosed herein) are observed during the treatment.

IV. Kits for Use in Treatment of Diseases Associated with Galectin-9

The present disclosure also provides kits for use in treating or alleviating a hematologic malignancy such as those disclosed herein (e.g., AML, MDS, or ALL). Such kits can include one or more containers comprising an anti-Gal9 antibody, e.g., any of those described herein (e.g., G9.2-17(IgG4), and optionally a second therapeutic agent (e.g., a chemotherapeutic agent as disclosed herein) to be co-used with the anti-Gal9 antibody, which is also described herein.

In some embodiments, the kit can comprise instructions for use in accordance with any of the methods described herein. The included instructions can comprise a description of administration of the anti-Gal9 antibody, and optionally the second therapeutic agent, to treat, delay the onset, or alleviate a target disease as those described herein. In some embodiments, the kit further comprises a description of selecting an individual suitable for treatment based on identifying whether that individual has the target disease, e.g., applying the diagnostic method as described herein. In still other embodiments, the instructions comprise a description of administering an antibody to an individual at risk of the target disease.

The instructions relating to the use of an anti-Gal9 antibody generally include information as to dosage, dosing schedule, and route of administration for the intended treatment. The containers may be unit doses, bulk packages (e.g., multi-dose packages) or subunit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the target hematologic malignancy. In some embodiments, instructions are provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. In some embodiments, a kit has a sterile access port (for example the container may be an intravenous solution bag or a vial having a stopper pierceable by a hypodermic injection needle). In some embodiments, the container also has a sterile access port (for example the container is an intravenous solution bag or a vial having a stopper pierceable by a hypodermic injection needle). At least one active agent in the composition is an anti-Gal9 antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present invention employs, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: a Phase 1 Open-Label, Multi-Center Study of the Safety, Pharmacokinetics (PK), and Anti-Tumor Activity of G9.2-17(IgG4) in Patients with Relapsed/Refractory Acute Myeloid Leukemia (AML), or with Relapsed/Refractory, High-Risk Myelodysplastic Syndrome (MDS)

G9.2-17(IgG4) is a fully human immunoglobulin gamma (IgG)4 monoclonal antibody (mAb) targeting the carbohydrate recognition domain 2 (CRD2) of Galectin-9 (gal-9) protein and is being developed for the treatment of patients with relapsed and refractory solid tumors and hematologic malignancies. Gal-9 plays a critical regulatory role in the anti-tumor immune responses. Gal-9, which is overexpressed and/or secreted in many cancer types (solid tumors and hematologic malignancies), functions as an immunosuppressor, conferring immune privilege to tumor cells and disabling immune-mediated cancer attack by regulating macrophages, T cells, natural killer (NK) cells, and myeloid-derived suppressor cells (MDSCs), as well as hindering cancer cell susceptibility to cytotoxic T cell induced death. G9.2-17(IgG4) has been designed to induce blockade of gal-9 and to interfere with the immunosuppressive functions of gal-9, which can result in immune reactivation and inhibition/control of tumor growth.

G9.2-17(IgG4) is expected to benefit treatment of acute myeloid leukemia (AML) and myelodysplastic syndrome (MDS) such as relapsed and/or refractory AML or MDS in at least the following:
Favorable safety profile.
Potential immunomodulation peripherally and within the bone marrow which is to be monitored by PD and efficacy assessments.
Clinical improvement of patients.

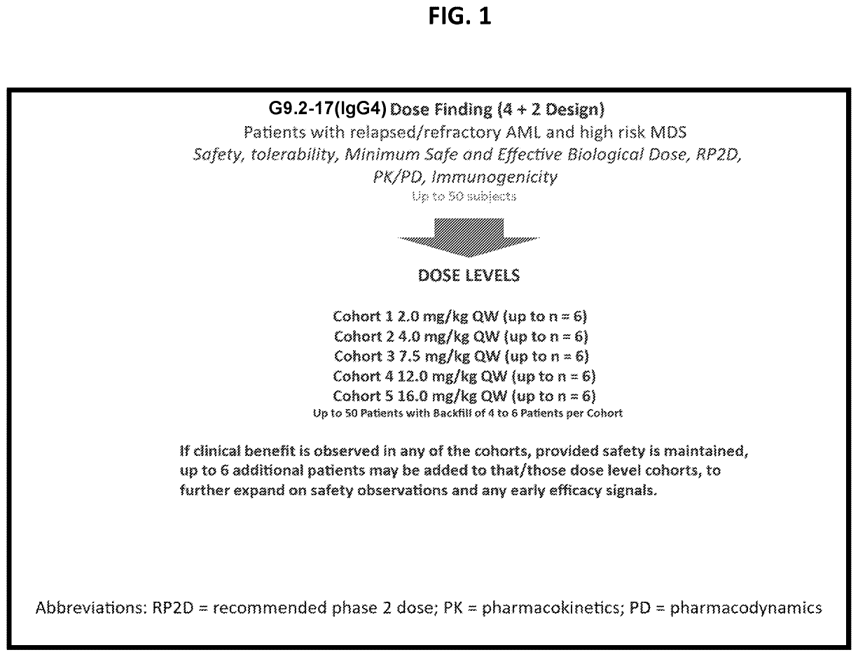

A study schematic is illustrated in FIG. 1. A summary of the study objectives and endpoints are provided in Table 3 below.

TABLE 3
Study Objectives and Endpoints

| | |
|---|---|
| Objectives | Endpoints |

Primary

| | |
|---|---|
| Establish the safety and tolerability of G9.2- | Evaluation of safety parameters including |
| 17(IgG4) as a single treatment | adverse events, vital sign measurements, |
| Confirm the dose or doses of G9.2- | clinical safety laboratory tests, 12-lead |
| 17(IgG4) for Phase 2 (RP2D) | electrocardiogram (ECG), |
| Determine the incidence of DLTs during | echocardiography/cardiac ultrasound |
| Cycle 1 (28 days) across dose levels | (ECHO), physical examinations |
| | Determination of MTD for G9.2-17(IgG4) |
| | Determination of the biologically effective |
| | dose of G9.2-17(IgG4) |
| | Determination of the RP2D dose or doses |
| | for G9.2-17(IgG4) |
| | Evaluation of DLTs during Cycle 1 |

Secondary

| | |
|---|---|
| Determine the preliminary efficacy of G9.2- | AML Endpoints: |
| 17(IgG4) as a single treatment | Rate of composite complete remission |
| | (CR + CR with incomplete hematological |
| | recovery [CRi]) |
| | Rate of Molecular Residual Disease (MRD) |
| | clearance among patients with: CR, CRi, |
| | CR + CRi, Morphologic Leukemia-free State |
| | (MLFS) |
| | Rate of CR |
| | Rate of PR |
| | Rate of Stable Disease (SD) |
| | Time-to-event Endpoints: |
| | Overall Survival (OS) |
| | Event-free survival (EFS) |
| | Cumulative incidence of relapse (CIR) |
| | Transfusion Independence: |
| | Rate of transfusion independence |
| | Duration of transfusion independence |
| | Hematological Improvement (HI): |
| | Erythroid (HI-E) |
| | Platelet (HI-P) |
| | Neutrophil (HI-N) |
| | Rate of patients achieving (MRD) negative |
| | status |
| | MDS Endpoints: |
| | Overall Response Rate (ORR): CR, |
| | Marrow CR, Partial Remission (PR), HI |
| | Rate of CR |
| | Rate of Marrow CR |
| | Rate of PR |
| | Rate of SD |
| | Durability of response (CR, PR and |
| | morphologic complete remission [mCR] |
| | rate) |
| | Cytogenetic Response: |
| | Complete |
| | Partial |
| | Survival Endpoints: |
| | OS |
| | EFS |
| | Transfusion Independence |
| | Rate of transfusion independence |
| | Duration of transfusion |
| | independence |
| | Hematological Improvement (HI): |
| | HI-E |
| | HI-P |
| | HI-N |

Exploratory

| | |
|---|---|
| Further evaluate the efficacy of AML | Rate of Progressive Disease |
| | Rate of Treatment Failure |
| | Death in Aplasia |
| | Death from Indeterminate Cause |
| | Rate of Relapse |
| | Hematological Relapse |
| | Molecular Relapse |
| Further evaluate the efficacy of MDS | Rate of Progressive Disease |
| | Rate of Treatment Failure |
| | Rate of Relapse |
| | Molecular Relapse |
| | Hematologic Relapse |
| Study pharmacodynamic markers, including | Multiplexing of serum/plasma |
| immunological and molecular changes in | cytokines |
| the peripheral blood and bone marrow, and | Mass cytometry whole blood |
| serum gal-9 | immune profiling |
| | Gene array from whole blood |
| | Gal-9 in serum by ELISA |
| Assess the immunogenicity of G9.2- | Evaluation of anti-drug antibodies (ADA) |
| 17(IgG4) | |

### I. Study Objectives

### Primary Objectives

Establish the safety and tolerability of G9.2-17(IgG4) as a single agent
Confirm the dose or doses of G9.2-17(IgG4) for Phase 2 (R2PD)
Determine the incidence of DLTs during Cycle 1 (28 days) across dose levels

### Secondary Objectives

Determine the preliminary efficacy of G9.2-17(IgG4) as a single treatment
Characterize the PK profile of G9.2-17(IgG4)

### Exploratory Objectives

Study pharmacodynamic markers, including immunological and molecular changes in the peripheral blood and bone marrow, and serum Gal-9
Assess the immunogenicity of G9.2-17(IgG4)

### II. Study Endpoints

### Primary Endpoints

Evaluation of safety parameters including adverse events, vital sign measurements, clinical safety laboratory tests, 12-lead electrocardiogram (ECG), echocardiography/cardiac ultrasound (ECHO), physical examinations
Determination of the biologically effective dose for G9.2-17(IgG4)
Determination of the maximum tolerated dose (MTD) for G9.2-17(IgG4)
Determination of the RP2D dose or doses for G9.2-17(IgG4)
Evaluation of DLTs during Cycle 1

### Secondary Endpoints

The secondary endpoints to determine preliminary efficacy (disease response) are as defined in Tables 4 and 5 below:

TABLE 4
AML Efficacy Endpoints and Definitions

| | |
|---|---|
| Efficacy Endpoint | Definition |

Natural History

| | |
|---|---|
| Rate of composite complete | CR: |
| remission (CR + CR with | Bone marrow blasts ≤5% |
| incomplete hematological | Absence of circulating blasts with Auer rods |
| recovery (CRi)) | Absence of extramedullary disease |
| | Platelets ≥100 × 10⁹/L |
| | ANC ≥1 × 10⁹/L |
| | RBC transfusion independent |
| | CRi |
| | All CR criteria and transfusion independence but with persistence |
| | of neutropenia, i.e., absolute neutrophil count of <1 × 10⁹/L and/or |
| | platelet count <100 × 10⁹/L |
| Rate of Molecular Residual | MRD: |
| Disease (MRD) clearance among | RT-qPCR (genetic lesions) |
| patients with: CR, CRi, CR + | MFC (immunophenotype markers) |
| CRi, Morphologic Leukemia-free | MLFS: |
| State (MLFS) | Bone marrow blasts ≤5% |
| | Absence of blasts with Auer rods |
| | Absence of extramedullary disease |
| | No hematologic recovery required |
| | CR: defined above |
| | CRi: defined above |
| Rate of CR | CR: defined above |
| Overall Remission Rate (ORR = | See above for definitions of CR, CRi, MLFS |
| CR + CRi + Rate of Partial | PR: |
| Remission (PR) + MLFS) | All hematologic criteria of CR |
| | Decrease of pretreatment bone marrow blast percentage by at least |
| | half to a value 5% to 25% (if the pretreatment bone marrow blast % |
| | was 20% to <50%) |
| Rate of Stable Disease (SD) | Absence of CR_{MRD}, CR, CRᵢ, PR, MLFS, and criteria for PD not |
| | met |
| Time-to-event Endpoints: | OS: |
| Overall Survival (OS) | Time to death from any cause, measured from the date of entry into |
| EFS | the trial to the date of death from any cause; patients not known to |
| Cumulative incidence of | have died at last follow-up are censored on the date they were last |
| relapse (CIR) | known to be alive |
| | EFS: |
| | Measured from the date of entry into the trial to the date of |
| | refractory disease, or relapse from CR or CRᵢ, or death from any |
| | cause; |
| | CIR: |
| | Defined for all patients achieving CR or CR_{i;,} measured from the |
| | date of achievement of a remission until the date of relapse; patients |
| | not known to have relapsed are censored on the date they were last |
| | examined; patients who died without relapse are counted as a |
| | competing cause of failure |
| Transfusion Independence: | Transfusion Independence for minimum of 8 weeks |
| Rate of Transfusion | Overall duration of transfusion independence |
| Independence | |
| Duration of Transfusion | |
| Independence | |

Hematological Improvement (HI)

| | |
|---|---|
| Erythroid (HI-E) | HI-E (pretreatment <11 g/dL): |
| Platelet (HI-P) | Hgb increased by ≥1.5 g/dL |
| Neutrophil (HI-N) | Relevant reductions of units of red blood cells (RBC) transfusions |
| Proportion of patients who | by an absolute number of at least 4 RBC transfusions/8 weeks |
| achieve HI, transfusion | compared with pretreatment transfusion number in the previous 8 |
| independence or CR | weeks. Only RBC transfusions given for an Hgb ≤9.0 g/dL |
| Duration of response for | pretreatment count in the RBC transfusion response evaluation |
| participants who achieved HI | HI-P (pretreatment <100 × 10⁹/L): |
| or better | Absolute increase of ≥30 × 10⁹/L for patients starting with >30 × |
| Percentage of patients with | 10⁹/L platelets |
| clinical benefit and/or | Increase from <20 × 10⁹/L >20 × 10⁹/L and by at least 100% |
| absence of hematologic | HI-N (pretreatment <1.0 × 10⁹/L): |
| progression per Investigator | At least 100% increase and an absolute increase >0.5 × 10⁹/L |
| assessment | |

Molecular Residual Disease (MRD)

| | |
|---|---|
| Rate of patients achieving MRD | MRD negativity |
| negative status | Calculated for subjects MRD positive at baseline |

TABLE 5
MDS Efficacy Endpoints and Definitions

| | |
|---|---|
| Efficacy Endpoint | Definition |

Natural History

| | |
|---|---|
| Overall Response Rate (ORR: | CR: |
| CR, Marrow CR) PR, HI | Bone marrow ≤5% myeloblasts with normal maturation of all cell |
| | lines |
| | Persistent dysplasia is noted |
| | Hgb ≥11 g/dL |
| | Platelets ≥100 × 10⁹/L |
| | Neutrophils ≥1 × 10⁹/L |
| | Blasts 0% |
| | Marrow CR: |
| | Bone marrow ≤5% myeloblasts and ≥50% decrease over |
| | pretreatment |
| | Peripheral blood: |
| | Marrow CR should be noted as a response with or without HI |
| | PR: |
| | All CR if abnormal before treatment except: |
| | Bone marrow blasts decreased by ≥50% over |
| | pretreatment but still >5% |
| | Cellularity and morphology not relevant |
| | HI: defined above |
| Rate of CR | Defined above |
| Rate of CR Marrow | Defined above |
| Rate of PR | Defined above |
| Rate SD | Failure to achieve PR, but no evidence of progression for >8 |
| | weeks |
| Durability of response (CR, PR | Time from response to relapse |
| and morphologic complete | |
| remission (mCR) rate) | |
| Cytogenetic Response | Complete: |
| | For patients with chromosome abnormality, disappearance of the |
| | chromosome abnormality without appearance of new ones |
| | Response must last at least 4 weeks |
| | Partial: |
| | At least 50% reduction of the chromosome abnormality |
| | Response must last at 4 weeks |
| Survival Endpoints: | OS: |
| OS | The date of death from any cause, measured from the date of entry |
| EFS | into the trial to the date of death from any cause; patients not |
| | known to have died at last follow-up are censored on the date they |
| | were last known to be alive |
| | EFS: |
| | Measured from the date of entry into the trial to the date of |
| | treatment failure or death from any cause |
| Transfusion Independence | Transfusion independence for MDS patients |
| | Duration of transfusion independence minimum of 8 weeks |

Hematological Improvement

| | |
|---|---|
| Erythroid (HI-E) | HI-E (pretreatment <11 g/dL): |
| Platelet (HI-P) | Hgb increased by ≥1.5 g/dL |
| Neutrophil (HI-N) | Relevant reductions of units of red blood cells (RBC) transfusions |
| Proportion of patients who | by an absolute number of at least 4 RBC transfusions/8 weeks |
| achieve HI, transfusion | compared with pretreatment transfusion number in the previous 8 |
| independence or CR | weeks. Only RBC transfusions given for an Hgb ≤9.0 g/dL |
| Duration of response for | pretreatment count in the RBC transfusion response evaluation |
| participants who achieved HI | HI-P (pretreatment <100 × 10⁹/L): |
| or better | Absolute increase of ≥30 × 10⁹/L for patients starting with >20 × |
| Percentage of patients with | 10⁹/L platelets |
| clinical benefit and/or | Increase from <20 × 10⁹/L >20 × 10⁹/L and by at least 100% |
| absence of hematologic | HI-N (pretreatment <1.0 × 10⁹/L): |
| progression per Investigator | At least 100% increase and an absolute increase >0.5 × 10⁹/L |
| assessment | |

### PK Endpoints

The secondary PK endpoints include evaluation of PK parameters of G9.2-17(IgG4) (including but not limited to area under the curve from time zero until 168 h [AUC_{0-168 h}], maximum observed serum concentration [Cₘₐₓ] and time to reach Cₘₐₓ [Tₘₐₓ]), and trough serum concentration [C_{trough}].

### Exploratory Endpoints

Exploratory endpoints are provided in Tables 6 and 7 below.

TABLE 6
Exploratory Efficacy Endpoints of AML

| | |
|---|---|
| Rate of | PD: Evidence for increase in bone marrow blast percentage and/or absolute increase in |
| Progressive | blast count in the blood: |
| Disease (PD) | >50% increase in bone marrow blasts over baseline (a minimum 15% point |
| | increase is required in cases with <30% blasts at baseline), or persistent marrow |
| | blast percentage of >70% over at least 3 months, without at least a 100% |
| | improvement in ANC to an absolute level (>0.5 × 10⁹/L), and/or platelet |
| | count to >50 × 10⁹/L, non-transfused, or |
| | >50% increase in peripheral blasts (WBC × % blasts) to >25 × 10⁹/L (in the |
| | absence of differentiation syndrome) |
| | New extramedullary disease |
| Rate of Treatment | Death in aplasia: |
| Failure: | Death occurring ≥7 days following completion of initial treatment while |
| Death in aplasia | cytopenic, with an aplastic or hypoplastic bone marrow obtained within 7 days of |
| Death from | death, without evidence of persistent leukemia |
| indeterminate | Death from indeterminate cause: |
| cause | Death occurring before completion of therapy, but no bone marrow examination |
| | available |
| | Death occurring ≥7 days following completion of initial therapy with no blasts in |
| | the blood, but no bone marrow examination available |
| Rate of Relapse | Molecular relapse |
| | Progression or relapse after HI: |
| | Proportion of patients who achieve HI, transfusion independence or CR |
| | Duration of response for participants who achieved HI or better |
| | Percentage of patients with clinical benefit and/or absence of hematologic |
| | progression |

TABLE 7
Exploratory Efficacy Endpoints of MDS

| | |
|---|---|
| Rate of PD | Patients with: |
| | <5% blasts: ≥50% increase in blasts to >5% blasts |
| | 5% to 10% blasts: ≥50% increase in blasts to >10% blasts |
| | 10% to 20% blasts: ≥50% increase in blasts to >20% blasts |
| | 20% to 30% blasts: ≥50% increase in blasts to >30% blasts |
| | Any of the following: |
| | At least 50% decrease from maximum remission/response in granulocytes or |
| | platelets |
| | Reduction in Hgb by >2 g/dL |
| | Transfusion dependence |
| Rate of Treatment | Death during treatment or disease progression characterized by worsening of |
| Failure | cytopenia increase in percentage of bone marrow blasts, or |
| | Progression to a more advanced MDS FAB subtype than pretreatment |
| | Primary refractory disease |
| | Death in aplasia |
| | Death from indeterminate cause |
| Rate of Relapse | Molecular relapse |
| | Progression or relapse after HI: |
| | Proportion of patients who achieve HI, transfusion independence or CR |
| | Duration of response for participants who achieved HI or better |
| | Percentage of patients with clinical benefit and/or absence of hematologic |
| | progression per Investigator assessment |

### Exploratory Immunological and Molecular Changes in Peripheral Blood and Bone Marrow Endpoints

Multiplexing of serum/plasma cytokines
Mass cytometry whole blood immune profiling
Gene array from whole blood
Gal-9 in serum by ELISA

### Exploratory Immunogenicity Endpoints

Evaluation of ADA

### III. Study Design

This is an open-label, non-randomized, multi-center, Phase 1, dose escalation study in patients with AML whose disease is relapsed/refractory to at least one line of prior therapy, with or without an allogeneic stem cell transplant, or in patients with a documented diagnosis of relapsed/refractory, high-risk MDS post at least one line of treatment and for whom no standard therapy that may provide clinical benefit is available. The 4+2 algorithm-based dose-escalation design (Lin et al., Biostatistics, 2001; Wheeler et al., PLoS One, 2016) is used to establish DLTs and to help identify the RP2D. This study is conducted in approximately 15 sites in the United States. The study duration is estimated to be 24 months. Follow-up for survival continues for up to 2 years.

The study design is outlined in FIG. 1, and the visit schedule and planned assessments at each visit are detailed in Table 8.

### Dose Escalation and De-escalation

### Treatment Period

This study consists of the following periods:
Screening period: Up to 2 weeks prior to first dose (Day −14 to Day −1)
Treatment period: 28-day treatment cycles as presented in the Schedule of Assessments (SoA) (Table 8)
Post treatment period: 30 days after last treatment (End of Treatment Visit/Early Termination Visit)
Follow-up period: Long term follow-up for up to 2 years (every 3 months)

### Dose Escalation

This dose-finding study is conducted using a 4+2 algorithm-based dose-escalation design (Lin et al., Biostatistics, 2001; Wheeler et al., PLoS One, 2016) to determine MTD or the biologically active dose that can be used to help identify the RP2D dose or doses. The definition of a biologically effective dose is the dose at which 0-100% of 6 patients in a cohort experience:
For AML: either molecular residual disease (MRD) negative status or reduction in bone marrow blasts to ≤5% with absence of circulating blasts with Auer rods.
For MDS: either Transfusion independence duration minimum of 8 weeks or reduction in bone marrow myeloblasts to ≤5%.

Up to 6 patients per treatment Cohorts 1-5 receive 60-minute intravenous (IV) infusions of G9.2-17(IgG4) every week (QW) on Day 1, Day 8, Day 15, and Day 22 of each 28-day cycle, starting at a dose of 2.0 mg/kg.

Patients assigned to a specific dose escalation cohort receive the corresponding study dose for that cohort. They receive study drug until progression of disease, unacceptable toxicity, or withdrawal from the study for other reasons. Patients who withdraw for reasons other than toxicity or tolerability issues during the first treatment cycle only are replaced.

TABLE 8
Schedule of Assessments

| | | Treatment Phase | | | | | | | | | | | | | | | | | | |
|---|---|---|---|---|---|---|---|---|---|---|---|---|---|---|---|---|---|---|---|---|
| | | | | | | | | | | | | | | | | | | | Post-Treatment | |
| | | | | | | | | | | | | | | | Cycle 4 | | | | End of | |
| | | Cycle 1 | | | | | Cycle 2 | | | | Cycle 3 | | | | (28 days) | | | | Treatment | Long Term |
| | | (28 days) | | | | | (28 days) | | | | (28 days) | | | | and beyond | | | | or Early | Follow- |
| | | Study Day | | | | | | | | | | | | | | | | | Termination | Up |
| | Pre-Dose | 1 | 2 | 8 | 15 | 22 | 1 | 8 | 15 | 22 | 1 | 8 | 15 | 22 | 1 | 8 | 15 | 22 | 30 days | (2 year) |
| | Screening | Cycle Day | | | | | | | | | | | | | | | | | after | Every |
| | −14 | C1 | C1 | C1 | C1 | C1 | C2 | C2 | C2 | C2 | C3 | C3 | C3 | C3 | CX | CX | CX | CX | last dose | 3 months |
| | to −1 | D1 | D2 | D8 | D15 | D22 | D1 | D8 | D15 | D22 | D1 | D8 | D15 | D22 | D1 | D8 | D15 | D22 | — | — |
| +/− days | | | | 1 | 1 | 1 | 1 | 1 | 1 | 1 | 1 | 1 | 1 | 1 | 1 | 1 | 1 | 1 | 3 | n/a |
| allowable | | | | | | | | | | | | | | | | | | | | |
| Previous and | X | X | X | X | X | X | X | X | X | X | X | X | X | X | X | X | X | X | X | |
| concomitant | | | | | | | | | | | | | | | | | | | | |
| medications ^{D} | | | | | | | | | | | | | | | | | | | | |
| AEs ^{E} | X | X | X | X | X | X | X | X | X | X | X | X | X | X | X | X | X | X | X | |
| ECHO/MUGA ^{F} | X | | | | | | | | | | | | | | X | | | | | |
| 12-lead ECG | X | | | | | | X | | | | | | | | | | | | | |
| Pre-dose only | | | | | | | | | | | | | | | | | | | | |
| Physical exam^{G} | X | X | X | X | X | X | X | X | X | X | X | X | X | X | X | X | X | X | X | |
| ECOG | X | X | | X | X | X | | X | X | X | X | X | X | X | | X | X | | X | |
| Vital Signs^{H} | X | X | X | X | X | X | X | X | X | X | X | X | X | X | X | X | X | X | X | |
| Bone marrow | | | | | | | | | | | | | | | | | | | | |
| aspiration | | | | | | | | | | | | | | | | | | | | |
| and/or biopsy^{I} | | | | | | | | | | | | | | | | | | | | |
| +/− days | | | | 1 | 1 | 1 | 1 | 1 | 1 | 1 | 1 | 1 | 1 | 1 | 1 | 1 | 1 | 1 | 3 | n/a |
| allowable | | | | | | | | | | | | | | | | | | | | |
| Pregnancy test ^{J} | X | | | | | | X | | | | X | | | | X | | | | X | |
| Hematology ^{K} | X | X | X | X | X | X | X | X | X | X | X | X | X | X | X | | X | X | X | |
| Serum | X | X | X | X | X | X | X | X | X | X | X | X | X | X | X | X | X | X | X | |
| Chemistry ^{L} | | | | | | | | | | | | | | | | | | | | |
| TSH, Free T4, | X | | | | | | X | | | | X | | | | X | | | | X | |
| scrum lipasc, | | | | | | | | | | | | | | | | | | | | |
| amylase, PTH, | | | | | | | | | | | | | | | | | | | | |
| FSH, LH, free | | | | | | | | | | | | | | | | | | | | |
| cortisol | | | | | | | | | | | | | | | | | | | | |
| Blood | X | X | | X | X | X | X | X | X | X | X | X | X | X | X | X | X | X | X | |
| Coagulation ^{M} | | | | | | | | | | | | | | | | | | | | |
| Urinalysis ^{N} | X | X | | | | | X | | | | X | | | | X | | | | X | |
| +/− days | | | | 1 | 1 | 1 | 1 | 1 | 1 | 1 | 1 | 1 | 1 | 1 | 1 | 1 | 1 | 1 | 3 | n/a |
| allowable | | | | | | | | | | | | | | | | | | | | |
| PD biomarkers | | X | | | X | | X | | X | | X | | | | X | | | | | |
| blood sampling^{O} | | | | | | | | | | | | | | | | | | | | |
| Hematology | X | X | | X | X | X | X | X | X | X | X | X | X | X | X | X | X | X | X | |
| blood film | | | | | | | | | | | | | | | | | | | | |
| (local)^{P} | | | | | | | | | | | | | | | | | | | | |
| Central MRD | | X | | | X | | X | | X | | X | | X | | X | | X | | X | |
| blood sampling^{Q} | | | | | | | | | | | | | | | | | | | | |
| PK blood | | X | X | X | X | X | X | | | | X | X | X | X | X | | | | | |
| sampling^{R} | | | | | | | | | | | | | | | | | | | | |
| Immunogenicity | | X | | | X | | X | | | | X | | | | X | | | | X | |
| (ADA)^{S} | | | | | | | | | | | | | | | | | | | | |
| +/− days | | | | 1 | 1 | 1 | 1 | 1 | 1 | 1 | 1 | 1 | 1 | 1 | 1 | 1 | 1 | 1 | 3 | n/a |
| allowable | | | | | | | | | | | | | | | | | | | | |
| Survival | | | | | | | | | | | | | | | | | | | | X |
| Follow-up | | | | | | | | | | | | | | | | | | | | |

ADA: anti-drug antibodies; AE: adverse event; ALT: alanine aminotransferase; APTT: activated partial thromboplastin time; AST: aspartate aminotransferase; C: cycle; CPK: creatine phosphokinase; COVID-19: Coronavirus SARS-CoV-2; CRP: C-reactive protein; D or d: day(s); ECG: electrocardiogram; ECOG: Eastern Cooperative Oncology Group; ECHO: echocardiography/cardiac ultrasound; FSH: follicle stimulating hormone; IMAR: immune-mediated adverse reaction; INR: international normalized ratio; LDH: lactate dehydrogenase; LH: luteinizing hormone; min: minute(s); MRD; molecular residual disease: MUGA: multigated acquisition scan; PD: pharmacodynamics; PK: pharmacokinetics; PT: prothrombin time; PTH: parathyroid hormone; PTT: partial thromboplastin time; QTcF: QT interval, Fridericia's Correction Formula; RBC: red blood cell count; SAE; serious adverse event; SGOT: serum glutamic-oxaloacetic transaminase; SGPT: serum glutamic pyruvic transaminase; TSH: thyroid stimulating hormone; WBC: white blood cell count.
A) Study drug administration: G9.2-17(IgG4) treatment will be administered, on CXD1, CXD8, CXD15, and CXD22 on every weekly cycle.
B) Demographics: Data include age, gender, racc, and ethnicity.
C) Medical history: In addition to general medical history, data collection also includes oncology history, surgical/transplant and radiation therapy history, and COVID-19 history and testing.
^{D} Previous and concomitant medications (including vaccines and complementary treatments/supplements): Data to include name, indication, dose, route, start and end dates for each. Allergies and intolerances and dose modifications while on study should also be obtained.
^{E} Adverse events: Any AEs starting or worsening after informed consent will be recorded. AEs should be followed until resolved to one of the following: baseline, stabilized, or deemed irreversible. All AEs, including SAEs, are to be collected after the first dose of study drug until 30 days after last dose of study medication. An SAE related to study procedures or study conduct must be reported to PureTech Health if it occurs prior to first dose of study drugs. SAEs must be reported as described herein. Treatment-related AEs ongoing at the post-treatment visit should be followed to resolution or until the Investigator considers them “chronic” or “stable.”
^{F}ECHO/MUGA: This assessment of heart function is conducted at Screening and repeated on Day 1 of Cycle 4; the assessment window is ±5 days. It should be conducted more frequently when clinically indicated and once every 3 months.
^{G}Physical exam: Include height at screening for determination of body surface area. Include weight at all scheduled exam times. A neurological exam will be conducted only on patients who have stable and/or pre-treated brain metastases.
^{H} Vital Signs: temperature, heart rate, blood pressure, respiratory rate.
Note:
To monitor for infusion reactions on Cycle 1 Day 1, vital signs must be recorded every 15 minutes during the 1-hour infusion of G9.2-17(IgG4), and every 30 minutes through 1-hour post-infusion, and hourly thereafter if applicable.
^{I}Bone marrow aspiration and/or biopsy: Monthly for the first 3 months (screening and then Day 1, Cycles 2 and 3 [±3 days]), then Q3M thereafter. Bone marrow aspirate to be sent to central lab for MRD analysis. Bone marrow aspirate is to be sent to the central lab for MRD analysis.
^{J} Pregnancy test (blood or urine): Only for women of childbearing potential with uterus in situ. Test results must be available before scheduled dosing.
^{K} Hematology: Analysis includes complete blood count, differential, platelets, hemoglobin. Collect blood samples pre-dose.
Note:
Cycle 1 Day 2 sampling has been added to provide for additional monitoring of Tumor Lysis Syndrome (TLS) laboratory parameters.
^{L} Serum chemistry: Analysis includes albumin, alkaline phosphatase, bilirubin (total, direct), blood urea nitrogen, calcium, CPK, creatinine, electrolytes (sodium, potassium, chloride, magnesium, phosphorus), gamma glutamyl transferase (gamma GT), glucose, hemoglobin A1c (HgbA1c) (only if history of Type 1 or Type 2 diabetes mellitus), SGPT (ALT) or SGOT (AST), total protein. Fasting glucose to be assessed only if clinically indicated. Collect blood samples pre-dose.
Note:
Cycle 1 Day 2 sampling has been added to provide for additional monitoring of TLS laboratory parameters.
^{M} Blood Coagulation: Collect blood samples pre-dose. Analysis includes APTT, PT, PTT, and INR (if on allowable anticoagulants), CRP, and troponin.
^{N} Urinalysis: Analysis includes color, appearance, dipstick for specific gravity, protein, white blood cell-esterase, glucose, ketones, urobilinogen, nitrite, WBC, RBC, pH (urine culture and sensitivity to be run only if patient is clinically symptomatic).
Note:
Urinalysis may be omitted on C1 D1 if collected during screening.
^{O}PD Biomarkers:
^{P}Hematology blood film (local): minimum assessments to capture: % of blasts, morphology of blasts with a particular focus on whether Auer rods arc present, blood cell dysplasia.
^{Q}MRD assessment: will be conducted at the central lab by MFC (flow cytometry).
^{R}PK blood sampling: Cycle 1 and Cycle 3 Day 1: blood samples will be collected pre-dose, end of study drug infusion (EOI) and 1 h (±15 min) post-study drug administration. Cycle 1 Day 2 only a PK sample will also be collected. Cycle 1 and Cycle 3 Day 8, Day 15, and Day 22, blood samples will be collected pre-dose and at EOI only. Cycle 2 and every even cycle thereafter: blood samples will be collected Day 1 only and should occur pre-dose and at EOI. Blood samples for PK will not be collected on every odd cycle after Cycle 3. PK sampling may be updated based on emerging PK data in this study.
^{S}ADA blood sampling: Blood samples will be collected pre-dose on Day 1 of every cycle and on Cycle 1 Day 15.

A total of 5 dose levels are evaluated within the 4+2 design, with the starting dose of 2.0 mg/kg QW:
Dose Escalation Cohort 1=2.0 mg/kg QW (up to n=6)
Dose Escalation Cohort 2=4.0 mg/kg QW (up to n=6)
Dose Escalation Cohort 3=7.5 mg/kg QW (up to n=6)
Dose Escalation Cohort 4=12.0 mg/kg QW (up to n=6)
Dose Escalation Cohort 5=16.0 mg/kg QW (up to n=6)

One cycle (28 days) is 4 dose administrations of G9.2-17(IgG4).

Opening the next higher dose cohort is initiated based on analysis of patient safety data focusing on occurrences of DLTs, other relevant safety and available PK data from the previous cohorts. No dose level skipping is allowed.

Patients treated prior to identification of the RP2D are allowed to dose escalate up to the highest dose level once they have cleared the 28-day DLT period in the cohort they are originally enrolled in. Dose escalations may not occur in the middle of a cycle. Patients can continue to dose escalate to the highest approved dose level until they are discontinued for toxicity or disease progression, or for other reasons (e.g., a patient elects to discontinue from the study). They may only escalate after completing a full cycle on their current dose level and not mid cycle.

In the absence of demonstrated clinical benefit, the time on study drug is limited to 6 cycles, i.e., 6 months. If a patient has not achieved a minimum of a PR as clinical benefit, the patient is taken off study drug, even in the absence of unacceptable toxicity. If a patient has demonstrated at least a PR as clinical benefit within 6 cycles (6 months) of starting study drug, that patient may continue dosing beyond 6 months until disease progression, patient refusal, or unacceptable toxicity.

If a DLT occurs in any patient during the first 28 days of treatment, that patient is permanently discontinued from study drug administration.

For patients who experience toxicities (including IMARs) outside of the DLT window, dose reduction is allowed only if the Investigator assesses that clinical benefit is being derived and may continue to be derived at a lower dose level of G9.2-17(IgG4). The dose of G9.2-17(IgG4) is initially reduced by 50%, and potentially by a further reduction of 50%, as defined by the dose modification guidance provided in the protocol. No further dose reductions are allowed. In cases where patients withdraw for reasons other than toxicity or tolerability issues during the first treatment cycle only, they are replaced until the MTD has been identified.

### Dose De-Escalation (Cohort-1 Only)

During Cycle 1 of Cohort 1 (2.0 mg/kg G9.2-17(IgG4)) only, a new Cohort-1 may be opened at a lower dose of G9.2-17(IgG4) as follows:
If 2 or more patients reach DLT, then a lower dose Cohort-1 may be opened at a lower dose.
If any sign of clinical benefit is observed in Cohort 1 based on secondary, exploratory endpoints or reductions in other markers of disease activity such as bone marrow blasts decreased by ≥50% over pretreatment, a lower dose Cohort 1 may be added for the purposes of exploring dose response relationships that will factor into dose selection for further development. Selection of the lower dose will be based on available PK, PD data as well as markers of disease activity.

### Completion of 4+2 Algorithm-Based Dose-Escalation

The 4+2 algorithm-based dose-escalation is completed when one or more of the G9.2-17(IgG4) doses evaluated has been identified as the RP2D dose or doses.

### Dose-Limiting Toxicity Criteria

DLTs assessed in this trial are defined as a clinically significant hematologic and/or non-hematologic AE or abnormal laboratory value not clearly resulting from the underlying leukemia or extraneous causes occurring during the first cycle (28 days) on study. Any patient that experiences a DLT during the first 28 days of treatment is permanently discontinued from study drug administration.

A DLT is a toxicity that meets any of the following criteria:
Any death not clearly due to the underlying leukemia or extraneous causes
Indications of potential drug-induced liver injury (Hy's Law cases) as follows:
   ALT or AST >3×the upper limit of normal (ULN) with confirmation by repeat testing 24 hours later, AND
   Serum total bilirubin (TBL) >2×ULN with confirmation by repeat testing 24 hours later.
   No other explanation can be found for the elevated TBL and/or aminotransferases (ATs), such as viral hepatitis (A, B or C), alcoholic or autoimmune hepatitis, pre-existing or acute liver disease, gall bladder obstruction or bile duct disease, Gilbert syndrome, disease progression, or another medication capable of causing the observed effect
All Grade ≥4 hematologic toxicities not clearly resulting from the underlying leukemia or extraneous causes, lasting >7 days. Events that exceed beyond the 28-day DLT monitoring period is also counted. All Grade 3-5 non-hematologic toxicities not clearly resulting from the underlying leukemia or extraneous causes, with the following exception:
   Grade 3 fatigue, asthenia, fever, anorexia (Grade 3 anorexia can be excluded only if it does not result in hospitalization, tube feeding, or use of total parenteral nutrition), or constipation.
   Grade 3 nausea, vomiting or diarrhea not requiring tube feeding, total parenteral nutrition, or requiring or prolonging hospitalization.
   Infection, bleeding, or other expected direct complication of cytopenias due to active underlying leukemia.
   Grade 3 infusion reaction including cytokine release syndrome (CRS), if successfully managed and which resolves within 72 hours.
   Grade 3 or 4 TLS if it is successfully managed clinically and resolves within 7 days without end-organ damage.
   Grade 3 or 4 isolated electrolyte abnormalities that last <72 hours will not be considered a DLT.

### End of Study

End of study is defined at the point when the RP2D or doses have been identified, all patients have been treated with G9.2-17(IgG4) until confirmed disease progression, or come off treatment for other reasons, and all patients have completed OS follow-up.

Patients are followed for OS for up to 2 years following the last dose of G9.2-17(IgG4).

The end of the study is defined as the date of the last patient's last visit.

### Trial Stopping Rules

Dose-escalation phase of 4+2 design is completed when one of the doses from Cohorts 1 to 5 or intermittent dose, has been identified as the RP2D. The RP2D is determined based on the MTD, DLTs, PK, PD, and additional safety data observed in each dose level and any other factors the sponsor may consider. The trial is stopped for safety if the lower limit of an Agresti and Coull binomial CI for the lowest study dose level exceeds the target DLT rate (Agresti et al., The American Statistician, 1998).

### IV. Study Population

### Inclusion Criteria

1. Patients ≥18 years of age at the time of obtaining informed consent.
2. Patients with morphologically documented primary or secondary AML by the World Health Organization (WHO) criteria (Arber et al., Blood, 2016), whose disease is relapsed/refractory to at least one line of prior therapy, with or without an allogeneic stem cell transplant and for whom no standard therapy that may provide clinical benefit is available or for patients who decline available standard of care.
3. Patients with a documented diagnosis of high-risk myelodysplastic syndrome (MDS), whose disease is relapsed/refractory, post at least one line of treatment based on the revised International Prognostic Scoring System (IPSS-R) (Greenberg et al., Blood, 2012) and for whom no standard therapy that may provide clinical benefit is available.
4. Patients are able to understand, sign, and date the written informed consent form at screening visit prior to any protocol-specific procedures.
5. Patients are able and willing to comply with study procedures as per protocol, including bone marrow biopsies.
6. Patient has an ECOG performance status ≤2.
7. Patient's interval from prior treatment to time of study drug administration is at least 2 weeks for cytotoxic agents (except hydroxyurea given for cytoreduction), or at least 5 half-lives for prior experimental agents or noncytotoxic agents. Upon discussion with the Medical Monitor, shorter than stated washout period may be considered provided that the patient has recovered from any clinically relevant safety issue and recovered to Grade ≤1 toxicity from prior therapies.
8. Patient must meet the following criteria as indicated on the clinical laboratory tests:
   a. WBC count at the time of the first dose of <25,000/uL.
   b. Aspartate aminotransferase or alanine aminotransferase ≤3×ULN (≤5.0×ULN if considered to be due to leukemic involvement).
   c. Total bilirubin ≤2×ULN (≤3×ULN if considered to be due to leukemic involvement or Gilbert's syndrome).
   d. Cretinine clearance of 60 m/min.
9. Patient has minimum life expectancy ≥3 months.
10. Female patient must be either: i) of non-child bearing potential: post-menopausal (defined as at least 1 year without any menses) prior to screening, or documented surgically sterile or status post hysterectomy (at least 1 month prior to screening); ii) if of childbearing potential, must have a negative serum or urine pregnancy test at screening, and must use 2 forms of birth control (at least one of which must be a barrier method) starting at screening and throughout the study period and for 90 days after the final study drug administration.
11. Female patient must not be breastfeeding at screening and during the study period, and for 90 days after the final study drug administration.
12. Female patient must not donate ova starting at screening and throughout the study period, and for 90 days after the final study drug administration.
13. Male patient and their female spouse/partners who are of childbearing potential must be using highly effective contraception consisting of 2 forms of birth control (one of which must be a barrier method) starting at screening.

### Exclusion Criteria

A subject who meets any of the following exclusion criteria is not eligible for inclusion in the study:
1. Patient diagnosed with acute promyelocytic leukemia (APL).
2. Patient has active malignant tumors other than AML. Participants with a history of adequately treated malignancy for which no anticancer systemic therapy (namely chemotherapy, immunotherapy, small molecule inhibitor, radiotherapy, or surgery) is ongoing or required during the course of the study. Participants who are receiving adjuvant therapy such as hormone therapy or zoledronic acid or denosumab are eligible.
3. Patient has persistent non-hematological toxicities of ≥Grade 2 (CTCAE v5.0), with symptoms and objective findings from prior AML treatment (including chemotherapy, kinase inhibitors, immunotherapy, experimental agents, radiation, hematopoietic stem cell transplant [HSCT] or surgery).
4. Patient has had HSCT and meets any of the following: has undergone HSCT within the 6-month period prior to the first study dose; has ≥Grade 2 persistent non-hematological toxicity related to the transplant donor lymphocytes infusion.
5. Patient has active graft versus host disease (GVHD) and patients receiving inmmunosuppressive treatment for GVHD. Calcineurin inhibitors must be withdrawn for at least 4 weeks before Screening.
6. Patient with symptomatic central nervous system (CNS) involvement of leukemia or other CNS diseases related to underlying and secondary effects of malignancy.
7. Patient has disseminated intravascular coagulation abnormality (DIC).
8. Patient has had major surgery within 4 weeks prior to the first study dose.
9. Patient has had radiation therapy within 4 weeks prior to the first study dose.
10. Patient has congestive heart failure New York Heart Association (NYHA) class 3 or 4, or patient with a history of congestive heart failure NYHA class 3 or 4 in the past, unless a screening echocardiogram or multigated acquisition (MUGA) scan performed within 3 months prior to study entry results in a left ventricular ejection fraction (LVEF) that is ≥45%.
11. Patient has any clinically important abnormalities in: i) rhythm; ii) conduction; iii) morphology on the resting ECG (e.g., complete left bundle bunch block, third degree heart block, second degree heart block, PR interval >250 msec); and/or iv) any factors that increase the risk of QTc prolongation or risk of arrhythmic events, such as congenital long QT syndrome or family history of long QT syndrome, that are medically unmanaged, based on the Investigator's judgement.
12. Patient is known to have symptomatic active infection including any identified active COVID-19 infection.
13. Patient is known to have human immunodeficiency virus infection.
14. Patient has known active hepatitis B or C, or other active hepatic disorder.
15. Patient has any condition which, in the Investigator's opinion, makes the patient unsuitable for study participation.
16. Any other medical, psychological, or social condition that may interfere with study participation or compliance, or compromise patient safety in the opinion of the Investigator.
17. Patients unwilling or unable to comply with the protocol.
18. Live vaccine administered within 30 days prior to start of treatment.
19. History of severe hypersensitivity reactions to any monoclonal antibodies (mAbs) and/or their excipients.
20. Known inherited or acquired bleeding disorders.
21. Active bleeding.
22. Patients with new thrombosis, embolism, cerebral hemorrhage, or other diseases or a medical history within one year before enrollment.
23. Drug abuse or long-term alcohol abuse that would affect the evaluation results.
24. Active autoimmune disease requiring systemic therapy (except autoimmune hypothyroidism treatable with synthetic thyroid hormone).

### V. Description of Study Assessments

### Demographics and Other Screening Assessments

At screening, patient demographic data will be collected. These include age, gender, race, and ethnicity. Safety assessments that are also part of the screening assessments are described below.

### Medical History

The medical history includes oncology history, surgical/transplant history radiation therapy history, and COVID-19 history and testing.
Personal medical history, including prior treatments/surgeries, record of any implants in situ or past implants, prior and/or current use of medical devices, concomitant medications (name, indication, dose, route, start and end dates dose modifications if any and reason), pre-existing symptoms, and AEs), hereditary diseases at risk of based on family history and complete family history to the best knowledge of the patient.
Record of any dental work performed in the past 12 months.
For patients with previously resected pancreatic adenocarcinoma, record whether the primary tumor was localized to the head of pancreas, pancreatic body or the pancreatic tail.
Bowel habits/typical frequency and consistency
Record of any dietary requirements or preferences (for example, practice of a particular diet regimen: intermittent fasting, keto diet etc.)
Record of allergies past and present (allergen, severity).

### ECHO/MUGA

ECHO and/or MUGA is obtained at the timepoints indicated in the SoA (Table 8). If clinically indicated, the assessment is to be repeated once every 3 months.

### ECOG

ECOG performance status is assessed at the timepoints indicated in the SoA (Table 8) using the following grading:
Grade 0: Fully active, able to carry on all pre-disease performance without restriction
Grade 1: Restricted in physically strenuous activity but ambulatory and able to carry out work of a light or sedentary nature, e.g., light housework, office work
Grade 2: Ambulatory and capable of all self-care but unable to carry out any work activities. Up and about more than 50% of waking hours
Grade 3: Capable of only limited self-care, confined to bed or chair more than 50% of waking hours
Grade 4: Completely disabled. Cannot carry on any self-care. Totally confined to bed or chair
Grade 5: Dead

### Efficacy Assessments

### Efficacy Assessments (Secondary)

Efficacy assessments are derived from updated International Working group (IWG) and key opinion leader recommendations for AML and MDS. Efficacy endpoints and definitions for AML and MDS are provided herein. All endpoints for both AML and MDS are derived from 4 types of assessment, collected according to the SoA (Table 8):
Hematology (routine component of clinical laboratory evaluation; see below)
MRD
MRD comprises central multiparameter flow cytometry (MFC) in blood for immunophenotype markers, and is conducted according to the SoA (Table 8)
Bone marrow biopsy/aspirate
Bone marrow aspirates are subject to MRD analysis by RT-qPCR/NGS molecular genetic analysis, and lao also include MFC, according to the SoA (Table 8). Bone marrow biopsy is to be evaluated locally to assess the degree and nature of bone marrow cellularity.
After cycle 3, it is allowed to not complete the bone marrow biopsy/aspirate if not medically feasible.
Hematology blood smears
The hematology blood film (stained for myeloperoxidase [MPO], Sudan black, alpha naphthyl acetate esterase [NAE], and Periodic-acid Schiff [PAS]), is used to determine:
   % of blasts
   Morphology of blasts with a particular focus on whether Auer rods are present (which is typical for AML)
   Blood cell dysplasia
Hematology blood films will be conducted locally according to the SoA (Table 8).

### Safety Assessments

### Adverse Events

See disclosures below.

### Clinical Laboratory Evaluations

Patients have blood samples collected for routine clinical laboratory testing (approximately 5 mL at each timepoint), according to the SoA (Table 8); additional tests may be performed at any time during the study as determined necessary by the Investigator or required by local regulations. Protocol-specific requirements for inclusion or exclusion of patients are detailed above.

The clinical laboratory parameters are analyzed at the site's local laboratory. Laboratory assessments to be completed include hematology and serum chemistry and are defined as following:
Serum Chemistry: To include glucose, total protein, albumin, electrolytes [sodium, potassium, chloride, magnesium, phosphorus], calcium, bilirubin (total, direct), SGPT GT), lactate dehydrogenase (LDH), creatinine, hemoglobin A1c (HgbA1c) (only if history of Type 1 or Type 2 diabetes mellitus), blood urea nitrogen, creatine phosphokinase (CPK)
   Thyroid stimulating hormone (TSH), fT4, lipase, amylase, PTH, FSH, luteinizing hormone (LH), free cortisol additionally at specified visits
   Fasting glucose will be assessed only if clinically indicated
Hematology: To include complete blood count, differential, platelets, hemoglobin
Coagulation: To include PT and PTT, activated partial thromboplastin time (APTT) and INR (if on allowable anticoagulants) C-reactive protein (CRP), and troponin
Urinalysis: Patients will have urine samples collected for routine urinalysis. The urinalysis will include color, appearance, and dipstick for specific gravity, protein, white blood cell-esterase, glucose, ketones, urobilinogen, nitrite, WBC, RBC, and pH, and urine culture (if patient is clinically symptomatic).

Abnormal laboratory findings associated with the underlying disease are not considered clinically significant unless judged to be more severe than expected for the participant's condition.

All laboratory tests with values considered clinically significantly abnormal during participation in the study or within 30 days after the last dose of study drug should be repeated until the values return to normal or baseline or are no longer considered clinically significant.

If clinically significant values do not return to normal/baseline or Grade 1 within a period of time, the etiology should be identified.

All protocol-required laboratory tests must be conducted in accordance with the laboratory manual and the SoA (Table 8).

If laboratory values from non-protocol-specified laboratory tests performed at the institution's local laboratory require a change in participant management or are considered clinically significant (e.g., SAE or AE or dose modification), then the results must be recorded.

### 12-Lead Electrocardiogram

12-lead ECG are to be obtained as outlined in the SoA (Table 8) using an ECG machine that automatically calculates the heart rate and measures heart rate, PR interval, QRS duration, distance in time on the ECG tracing from the start of the QRS complex to the end of T-wave (QT) interval, and QTcF intervals.

### Vital Signs

Vital signs are to be measured in a post-supine position after 5 minutes rest and will include temperature, blood pressure (systolic and diastolic), heart rate, and respiratory rate, according to the SoA (Table 8).

To monitor for G9.2-17(IgG4) infusion reaction on Cycle 1 Day 1, vital signs must be recorded every 15 minutes during the 1-hour infusion of the antibody, and every 30 minutes through 1-hour post-infusion, and hourly thereafter if applicable.

### Physical Examination

Medical and physical examinations must be performed by a qualified physician, nurse practitioner, or physician assistant, and should include a thorough review of all body systems. Additionally, height is to be measured at screening only for determination of body surface area. Include weight at all scheduled exam times. A Neurological exam will be conducted only on patients who have stable and/or pre-treated brain metastases.

### Monitoring and Treatment for Tumor Lysis Syndrome

The following precautions are implemented to monitor and treat potential TLS:
In order to provide for adequate monitoring of laboratory parameters associated with TLS during the first week of G9.2-17(IgG4) treatment, in addition to the scheduled blood draw on Cycle D1 Day 1, a second blood draw will be collected on Cycle 1 Day 2.
Patients may be risk assessed for TLS prior to each G9.2-17(IgG4) infusion and, where clinically indicated (e.g., TLS experienced with prior therapy regimens), treated according to institutional standard of care, such as with adequate hydration and uricostatics prior to the start of the G9.2-17(IgG4) infusion.
Where a subject has a high disease burden and is at greater risk of TLS, it may also be appropriate to keep/admit them for additional monitoring following G9.2-17(IgG4) administration.

### Pharmacokinetics

The following serum PK parameters will be calculated for G9.2-17(IgG4), if possible:
AUC_{0-168 h}
Cₘₐₓ
Tₘₐₓ
Serum concentration vs. time profiles

Blood samples of approximately 5 mL are collected and processed to serum at each timepoint as specified in the SoA (Table 8). A complete list of PK parameters is to be provided in the statistical analysis plan.

### Pharmacodynamic Biomarkers

Planned time points for biomarker assessments are provided in the SoA (Table 8); sampling may be decreased to every 3^{rd} cycle after 6 months of treatment. The following samples for biomarker research are required and will be collected from all participants in this study as specified in the SoA.
Blood samples, to be collected prior to study drug administration (approximately 15 mL pre-dose).
Samples will be tested for PD biomarkers (by flow cytometry, ELISA, IHC, or multiplex phenotyping) to evaluate their association with the observed clinical responses to G9.2-17(IgG4) using validated assays.

The following biomarkers are planned to be assessed for in this study (note the listing below includes expected biomarkers but are not limited to only them):
Multiplexing of serum/plasma cytokines
Mass cytometry whole blood immune profiling
Gene array from whole blood
Gal-9 in serum by ELISA

### Immunogenicity Assessments

Blood samples (approximately 3 mL) are collected from all participants according to the SoA (Table 8) and processed to serum. Additionally, serum samples should also be collected at the End of Treatment/Early termination visit from patients who discontinued study drug or were withdrawn from the study.

### VI. Treatment

### Preparation, Handling, and Storage

G9.2-17(IgG4) is supplied in single use vials. G9.2-17(IgG4) liquid drug product is to be diluted to the target dose prior to administration. All dilutions should be performed in a controlled and sterile environment (patient dose is prepared for and delivered via an approximately 60 minutes IV infusion).

### Dose and Administration

All patients receive G9.2-17(IgG4). G9.2-17(IgG4) is administered via IV infusion for approximately 60 minutes, QW. The infusion starts and duration of infusion is recorded.

Patients receive G9.2-17(IgG4) as a single agent at sequentially increasing doses starting at 2.0 mg/kg, in accordance with the 4+2 design.

Patients who experience a DLT in Part 1 do not resume treatment.

### Dose Administration

If an infusion-related reaction is encountered, interrupt the infusion and if clinically indicated, administer relevant medication(s) (e.g., anti-histamine, anti-emetic, steroids, anti-pyretics, beta-blocker(s) etc.). If it is deemed appropriate to resume the infusion, resume at a slower infusion rate. Upon re-initiation of infusion, the new rate, as well as the stop and restart times for the infusion will be recorded.

For subsequent cycles for the same patient, apply the appropriate pre-medications (anti-histamine, anti-emetic, steroids, anti-pyretics, beta-blocker(s) etc., as clinically indicated) and consider utilizing a slower infusion rate.

### Dose Modification

The decision to proceed to the next dose level of G9.2-17(IgG4) is made based on safety, tolerability, and potential preliminary PK data obtained in at least 4 patients at the prior dose level. The dosing schedule may also be adjusted based on PK data obtained.

Detailed dose modification instructions are available in Tables 9 and 10:

TABLE 9
Management of Immune-Mediated Adverse Reactions (IMARs) Caused by G9.2-17(IgG4)

| IMAR | Guidance for Management |
|---|---|
| Immune-mediated | Monitor patients for signs with radiographic imaging and for clinical symptoms |
| pneumonitis | of pneumonitis. |
| | Withhold G9.2-17(IgG4) for moderate and permanently discontinue for severe |
| | or life-threatening pneumonitis. |
| | Administer corticosteroids at a dose of 1 to 2 mg/kg/day prednisone |
| | equivalents for moderate (Grade 2) or more severe (Grade 3-4) pneumonitis, |
| | followed by corticosteroid taper. |
| | Withhold G9.2-17(IgG4) until resolution to <Grade 2. For moderate (Grade 2) |
| | pneumonitis. Resume treatment at a 50% dose reduction. |
| | Upon recurrence of Grade 2 pneumonitis at the reduced dose of G9.2- |
| | 17(IgG4), permanently discontinue G9.2-17(IgG4). |
| Immune-mediated | Monitor patients for signs and symptoms of colitis. |
| colitis | Administer corticosteroids at a dose of 0.5 to 1 mg/kg/day prednisone |
| | equivalents followed by corticosteroid taper for moderate (Grade 2) colitis of |
| | more than 5 days duration. |
| | If worsening or no improvement occurs despite initiation of corticosteroids, |
| | increase dose to 1 to 2 mg/kg/day prednisone equivalents. |
| | When initial Grade 2 colitis resolves to <Grade 2, resume G9.2-17(IgG4) at a |
| | 50% dose reduction. |
| | If ≥Grade 2 colitis recurs, withhold G9.2-17(IgG4) until colitis resolves |
| | to <Grade 2. Resume G9.2-17(IgG4) at an additional 50% dose reduction. No |
| | further dose reductions for G9.2-17(IgG4) are allowed. |
| | Administer corticosteroids at a dose of 1 to 2 mg/kg/day prednisone |
| | equivalents followed by corticosteroid taper for severe (Grade 3) or life- |
| | threatening (Grade 4) colitis. |
| | When Grade 3 colitis resolves to ≤Grade 2, resume G9.2-17(IgG4) at a 50% |
| | dose reduction. |
| | Permanently discontinue G9.2-17(IgG4) for life-threatening (Grade 4) or for |
| | recurrent colitis of ≥Grade 2 upon re-initiation of G9.2-17(IgG4). |
| Immune-mediated | Monitor for changes in liver function. |
| hepatitis | Administer corticosteroids at a dose of 0.5 to 1 mg/kg/day prednisone |
| | equivalents for moderate (Grade 2) transaminase elevations. |
| | Withhold G9.2-17(IgG4) for moderate (Grade 2) immune-mediated hepatitis. |
| | When resolved to <Grade 2 resume G9.2-17(IgG4) at a 50% dose reduction. |
| | If ≥Grade 2 hepatitis recurs, permanently discontinue G9.2-17(IgG4). |
| | Administer corticosteroids at a dose of 1 to 2 mg/kg/day prednisone |
| | equivalents followed by corticosteroid taper for severe (Grade 3) or life- |
| | threatening (Grade 4) transaminase elevations, with or without concomitant |
| | elevation in total bilirubin. |
| | Permanently discontinue G9.2-17(IgG4) for severe (Grade 3) or life- |
| | threatening (Grade 4) immune-mediated hepatitis. |
| Immune-mediated | Administer hormone replacement as clinically indicated and corticosteroids at a |
| endocrinopathies: | dose of 1 mg/kg/day prednisone equivalents followed by corticosteroid taper |
| Hypophysitis | for moderate (Grade 2) or greater hypophysitis. |
| | Withhold G9.2-17(IgG4) for moderate (Grade 2) or severe (Grade 3) and when |
| | resolved to <Grade 2, resume G9.2-17(IgG4) at a 50% dose reduction. |
| | If ≥Grade 2 toxicity recurs, permanently discontinue G9.2-17(IgG4). |
| | Permanently discontinue G9.2-17(IgG4) for life-threatening (Grade 4) |
| | hypophysitis. |
| Adrenal insufficiency | Monitor patients for signs and symptoms of adrenal insufficiency. |
| | Withhold G9.2-17(IgG4) for moderate (Grade 2) and once resolved to <Grade |
| | 2, resume G9.2-17(IgG4) at a 50% dose reduction. |
| | If ≥Grade 2 toxicity recurs, permanently discontinue G9.2-17(IgG4). |
| | Administer corticosteroids at a dose of 1 to 2 mg/kg/day prednisone |
| | equivalents followed by a corticosteroid taper for severe (Grade 3) or life- |
| | threatening (Grade 4) adrenal insufficiency. |
| | Permanently discontinue G9.2-17(IgG4) for severe (Grade 3) or life- |
| | threatening (Grade 4) adrenal insufficiency, or for recurrent colitis of ≥Grade 2 |
| | upon re-initiation of G9.2-17(IgG4). |
| Hypothyroidism and | Monitor thyroid function prior to and periodically during tislelizumab |
| Hyperthyroidism | treatment. |
| | Administer hormone replacement therapy for hypothyroidism. |
| | Initiate medical management for control of hyperthyroidism. |
| | There are no recommended dose adjustments of G9.2-17(IgG4) for <Grade 3 |
| | hypothyroidism or hyperthyroidism. |
| | Discontinue G9.2-17(IgG4) for ≥Grade 3 hypothyroidism or hyperthyroidism. |
| Type 1 Diabetes | Withhold G9.2-17(IgG4) in cases of severe (Grade 3) hyperglycemia until |
| Mellitus | metabolic control is achieved. |
| | Upon resolution to ≤Grade 2, resume G9.2-17(IgG4) at a 50% dose reduction. |
| | If ≥Grade 2 toxicity recurs, permanently discontinue G9.2-17(IgG4). |
| | Permanently discontinue G9.2-17(IgG4) for life-threatening (Grade 4) |
| | hyperglycemia. |
| Immune-mediated | Monitor patients for elevated serum creatinine prior to and periodically during |
| nephritis and renal | treatment. |
| dysfunction | Administer corticosteroids at a dose of 0.5 to 1 mg/kg/day prednisone |
| (Defined as renal | equivalents for moderate (Grade 2) or severe (Grade 3) increased serum |
| dysfunction or ≥Grade | creatinine, if worsening or no improvement occurs, increase dose of |
| 2 increased creatinine, | corticosteroids to 1 to 2 mg/kg/day prednisone equivalents. |
| requirement for | Withhold G9.2-17(IgG4) for moderate (Grade 2) or severe (Grade 3) increased |
| corticosteroids, and no | serum creatinine. When resolved to <Grade 2, resume G9.2-17(IgG4) at a 50% |
| clear alternate | dose reduction. |
| etiology) | If ≥Grade 2 toxicity recurs, permanently discontinue G9.2-17(IgG4). |
| | Administer corticosteroids at a dose of 1 to 2 mg/kg/day prednisone |
| | equivalents followed by corticosteroid taper for life-threatening (Grade 4) |
| | increased serum creatinine. |
| | Permanently discontinue G9.2-17(IgG4) for life-threatening (Grade 4) |
| | increased serum creatinine. |
| Immune-mediated skin | Withhold for severe and permanently discontinue for life-threatening rash. |
| adverse reactions | For symptoms or signs of SJS or TEN, withhold G9.2-17(IgG4) and refer the |
| (Immune-mediated | patient for specialized care for assessment and treatment. |
| rash, including | If SJS or TEN is confirmed, permanently discontinue G9.2-17(IgG4). |
| Stevens-Johnson | For immune-mediated rash³ Grade 2, administer corticosteroids at a dose of 1 |
| syndrome (SJS) and | to 2 mg/kg/day prednisone equivalents followed by a corticosteroid taper for |
| toxic epidermal | severe (Grade 3) or life-threatening (Grade 4) rash. |
| necrolysis (TEN)) | Withhold G9.2-17(IgG4) for moderate (Grade 2) and severe (Grade 3) rash and |
| | when resolved to <Grade 2, resume G9.2-17(IgG4) at a 50% dose reduction. |
| | If ≥Grade 2 rash recurs, withhold G9.2-17(IgG4) until resolved to <Grade 2 |
| | and resume G9.2-17(IgG4) at an additional 50% dose reduction. No further |
| | dose reductions are allowed. |
| | Permanently discontinue G9.2-17(IgG4) for life-threatening (Grade 4) rash. |
| Immune-mediated | Monitor for changes in neurologic function. |
| encephalitis | Evaluation of patients with neurologic symptoms may include, but not be |
| | limited to, consultation with a neurologist, brain MRI, and lumbar puncture. |
| | Withhold G9.2-17(IgG4) in patients with new-onset moderate to severe |
| | neurologic signs or symptoms and evaluate to rule out infectious or other |
| | causes of moderate to severe neurologic deterioration. |
| | If other etiologies are ruled out, administer corticosteroids at a dose of 1 to 2 |
| | mg/kg/day prednisone equivalents for patients with immune-mediated |
| | encephalitis, followed by corticosteroid taper. |
| | Permanently discontinue G9.2-17(IgG4) for immune-mediated encephalitis. |
| Other IMARs | If uveitis occurs in combination with other IMARs, consider a Vogt-Koyanagi- |
| (May include: | Harada-like syndrome, and may require treatment with systemic steroids to |
| myocarditis, | reduce the risk of permanent vision loss. |
| rhabdomyolysis, | For any suspected IMARs, exclude other causes. |
| myositis, uveitis, iritis, | Based on the severity (Grade) of the adverse reaction, permanently discontinue |
| pancreatitis, facial and | or withhold G9.2-17(IgG4), administer high-dose corticosteroids, and if |
| abducens nerve | appropriate, initiate hormone replacement therapy. |
| paresis, demyelination, | Upon improvement to Grade 1 or less, initiate corticosteroid taper and continue |
| polymyalgia | to taper over at least 1 month. |
| rheumatica, | Consider restarting G9.2-17(IgG4) after completion of corticosteroid taper |
| autoimmune | based on the severity of the event, resuming at a 50% dose reduction. |
| neuropathy, Guillain- | |
| Barré syndrome, | |
| hypopituitarism, | |
| systemic inflammatory | |
| response syndrome, | |
| gastritis, duodenitis, | |
| sarcoidosis, histiocytic | |
| necrotizing | |
| lymphadenitis | |
| (Kikuchi | |
| lymphadenitis), motor | |
| dysfunction, vasculitis, | |
| aplastic anemia, | |
| pericarditis, and | |
| myasthenic syndrome.) | |

TABLE 10
Recommended Dose Modifications for G9.2-17(IgG4)
(AEs Outside the DLT Window and Other Than IMARs)

| Adverse Reaction | Severity | G9.2-17(IgG4) Dose Modification |
|---|---|---|
| Non-hematologic | ≥Grade 4 | Permanently discontinue treatment with |
| and hematologic | | G9.2-17(IgG4). |
| toxicities | | No dose reduction. |
| Hematologic | Grade 3 | |
| toxicities | | |
| Non-hematologic | Grade 3 | Withhold treatment until toxicity resolves |
| toxicities | | to ≤Grade 2. |
| (except those listed | | Resume G9.2-17(IgG4) at 50% reduction |
| here) | | from previous dose. |
| Management of | | If recurs again at ≥Grade 3 severity, hold |
| IMARs Caused by | | until resolved to ≤Grade 2 and implement |
| G9.2-17(IgG4)) | | a further 50% dose reduction. |
| | | No further dose reductions are allowed. |
| Gastrointestinal | Grade 3 | Withhold G9.2-17(IgG4) until toxicity |
| (Nausea, Vomiting) | Not requiring hospitalization | reverses to ≤Grade 2 |
| | or parenteral nutrition support; | Resume G9.2-17(IgG4) with no dose |
| | managed by supportive care | reduction |
| Electrolyte | Grade 3 corrected to ≤Grade | If recurrent, withhold G9.2-17(IgG4) until |
| Abnormalities | 2 within 24 hours | toxicity reverses to ≤Grade 2 |
| Electrolyte | ≥Grade 3 lasting <24-72 | Then resume G9.2-17(IgG4) at a 50% |
| Abnormalities | hours | reduction from the previous dose |
| | Not clinically complicated; | If recurrent, withhold G9.2-17(IgG4) until |
| | resolves spontaneously or | toxicity reverses to ≤Grade 2 |
| | responds to conventional | Resume G9.2-17(IgG4) at an additional |
| | medical interventions | 50% dose reduction |
| Amylase or Lipase | ≥Grade 3 | No further dose reductions are allowed |
| Elevation | Not associated with symptoms | |
| | or clinical manifestations of | |
| | pancreatitis | |

### Dose Delay

If any clinically meaningful AE of Grade ≥3 possibly related or related to one or more study drugs occurs, it is discussed before continuing with dosing. Where judged appropriate, a dose delay may be necessary for Grade 3 AE.

### Dose Reduction

No dose reductions are allowed for any patient who is being evaluated for DLTs (within the 28-day DLT window).

Dose reduction is allowed only if clinical benefit is being derived and may continue to be derived under dose reduced conditions, see Table 9 (for IMARs) or 10 (for other AEs).

### Dose Modification for IMARs

If an IMAR occurs, refer to Table 9 (IMARs) for guidance on dose management of G9.2-17(IgG4).

All relevant medical exam(s)/test(s) should be carried out in order to confirm that the adverse event is an IMAR.

### Discontinuation of Study Drug

In rare instances, it may be necessary for a patient to permanently discontinue study drug. If study drug is permanently discontinued due to reasons other than disease progression, and the patient is not being treated with other anticancer therapy(ies), the patient continues to be evaluated for disease progression for up to 2 years. See Table 8 (SoA) for data to be collected at the time of discontinuation of study drug and follow-up and for any further evaluations that need to be completed.

Every effort must be made by study personnel to keep patients on study treatment until one of the reasons for study treatment termination are met (disease progression, toxicity related to the study drug, withdrawal of consent). If the patient has radiographic progression but no unequivocal clinical progression and alternate treatment is not initiated, the patient may continue on study treatment at the Investigator's discretion. However, if patients have unequivocal clinical progression without radiographic progression, study treatment should be stopped as per Investigator's judgment and patients advised regarding available treatment options.

A patient may be discontinued prior to disease progression for any of the following reasons:
A DLT
An AE occurs/recurs outside of the DLT window that requires discontinuation of study treatment(s)
An IMAR occurs/recurs that requires discontinuation of study treatment(s)
Termination of the study by PureTech Health, LLC
Intercurrent illness or medical condition that prevents further administration of treatment or may jeopardize the patient's safety if they continue on study treatment
Due to Patient's or Investigator's decision
Pregnancy
Use of a non-protocol anticancer therapy
Significant deviation from protocol on the part of the patient (includes lack of compliance)
Significant protocol violation on the part of the Investigator

The explanation of why the patient is discontinuing study treatment should be documented in the CRF. If the patient discontinues study treatment due to toxicity, “Dose-Limiting Toxicity” or “Adverse Event” is recorded as the primary reason for withdrawal. If a patient is prematurely discontinued from the study at any time due to an AE or SAE, the patient must be followed until resolution to Grade 2 or less, unless it is unlikely to improve because of the underlying disease.

### Concomitant Therapies

Prior and concomitant medications, including vaccines and complementary treatments/supplements, will be documented for each patient at each scheduled visit (Table 8).

Any medication or vaccine (including over-the-counter or prescription medicines, recreational drugs, vitamins, and/or herbal supplements) that the participant is receiving at the time of enrollment or receives during the study must be recorded along with:
Reason for use
Dates of administration including start and end dates
Dosage information including dose and frequency

### Permitted Medications/Therapies

The following concomitant medications are allowed:
In patients on-study exceeding WBC counts of 25,000/uL. the use of hydroxyurea is permitted according to standard institutional practice for the first 2 cycles, concomitantly with G9.2-17(IgG4).
The Investigator should treat any of the following according to standard institutional practice:
   Infusion reactions
   Tumor lysis syndrome
   Cytokine release syndrome.
Continuation of bisphosphonate treatment (e.g., zoledronic acid) or denosumab for bone metastases, which have been stable for at least 6 months before treatment (C1D1).
The use of inhaled corticosteroids and mineralocorticoids (e.g., fludrocortisone), topical steroids, intranasal steroids, intra-articular steroids, and ophthalmic steroids.
Prophylactic or therapeutic use of anticoagulants.
Vaccination for COVID-19, common flu and/or other common clinically required indications (e.g., tetanus, pneumococcus, HBV, etc.) is allowed before or during the study period. The timing and type of vaccine must be recorded.
Closely monitor sensitive narrow therapeutic index CYP3A4 substrates such as, but not limited to, alfentanil, dihydroergotamine, ergotamine, fentanyl, pimozide, and quinidine and have doses adjusted as necessary given that G9.2-17(IgG4) can potently inhibit TGF-β and IL-10 in vitro. Reducing cytokine levels from patient baseline level may change CYP expression/activity and exposure for sensitive CYP3A4 substrates. Note: Immunosuppressant CYP3A4 substrates like tacrolimus, cyclosporine, everolimus, and sirolimus are prohibited.

### Prohibited Medications/Therapies

The following medications are not allowed while on this study:
Concomitant administration of investigational agents, other than G9.2-17(IgG4), for any indication.
Systemic immunosuppressive treatment, including, but not limited to cyclophosphamide, azathioprine, methotrexate, thalidomide, tacrolimus, cyclosporine, everolimus, sirolimus, and anti-TNF agents. However, patients are allowed to take acute, low dose systemic immunosuppressant medications (e.g., ≤10 mg/day of prednisone or equivalent).
Replacement therapy (e.g., thyroxine, insulin, physiologic corticosteroid replacement therapy [e.g., ≤10 mg/day of prednisone equivalent] for adrenal or pituitary insufficiency) is not considered a form of systemic treatment.

### VII. Adverse Events

### Definitions

### Adverse Events

An AE is any event, side effect, or other untoward medical occurrence that occurs in conjunction with the use of a medicinal product in humans, whether or not considered to have a causal relationship to this treatment. An AE can, therefore, be any unfavorable and unintended sign (that could include a clinically significant abnormal laboratory finding), symptom, or disease temporally associated with the use of a medicinal product, whether or not considered related to the medicinal product.

Events meeting the definition of an AE include:
Exacerbation of a chronic or intermittent pre-existing condition including either an increase in frequency and/or intensity of the condition
New conditions detected or diagnosed after study drug administration that occur during the reporting periods, even though it may have been present prior to the start of the study
Signs, symptoms, or the clinical sequelae of a suspected interaction
Signs, symptoms, or the clinical sequelae of a suspected overdose of either study drug or concomitant medications (overdose per se will not be reported as an AE/SAE)

Events that do not meet the definition of an AE include:
Medical or surgical procedure (e.g., endoscopy, appendectomy); the condition that leads to the procedure should be reported as an AE if it meets the criteria of an AE
Situations where an untoward medical occurrence did not occur (e.g., social and/or convenience admission to a hospital)
Anticipated day-to-day fluctuations of pre-existing disease(s) or condition(s) present or detected at the start of the study that do not worsen

If there is evidence of an AE through report or observation, the Investigator or designee evaluates further and records the following information:
Time of onset and resolution
Severity
Causality/relation to study treatment
Action taken regarding study drug
Outcome

### Serious Adverse Events

An SAE is any event that meets any of the following criteria:
Results in death.
Is life-threatening.
Requires inpatient hospitalization or prolongation of existing hospitalization.
Results in persistent or significant disability/incapacity.
Is a congenital anomaly/birth defect.
Is an important medical event that may not result in death, be life-threatening, or require hospitalization. The event is considered an SAE when, based upon appropriate medical and scientific judgment, the event may jeopardize the subject and may require medical or surgical intervention to prevent one of the outcomes listed above. Examples of such events include: intensive treatment in an emergency room or at home for allergic bronchospasm, blood dyscrasias or convulsions that do not result in inpatient hospitalization, or the development of drug dependency or drug abuse.

### Suspected Unexpected Serious Adverse Reactions

A suspected, unexpected serious adverse reaction (SUSAR) is an adverse event that is both unexpected (i.e., not listed in the Reference Safety Information for the investigational agent) and meets the definition of a serious adverse drug reaction, the specificity or severity of which is not consistent with those noted in the Reference Safety Information (i.e., the IB for an investigational agent).

### Clinical Laboratory Abnormalities and Other Abnormal Assessments

Abnormal laboratory findings (e.g., clinical chemistry, hematology, and urinalysis) or other abnormal assessments (e.g., ECGs or vital signs) that are judged as clinically significant are to be recorded as AEs and SAEs if they meet the definition of an AE or SAE. Clinically significant abnormal laboratory findings or other abnormal assessments that are detected during the study or are present at screening and significantly worsen following the start of the study will be reported as AEs or SAEs. However, clinically significant abnormal laboratory findings or other abnormal assessments that are associated with the disease being studied, unless judged by the Investigator as more severe than expected for the patient's condition, or that are present or detected at the start of the study and do not worsen, are not reported as AEs or SAEs.

Laboratory measurements that deviate clinically significantly from previous measurements (as determined by the Investigator) may be repeated. If warranted, additional or more frequent testing than is specified in the protocol should be done to provide adequate documentation of AEs and the resolution of AEs.

The Investigator exercises his or her medical and scientific judgment in deciding whether an abnormal laboratory finding or other abnormal assessment is clinically significant.

### Adverse Events of Special Interest

An AESI is an AE of scientific and medical concern specific to the sponsor's product or program, which requires additional monitoring. Such an event might warrant further investigation to better characterize and understand it.

For this study, IMARs and IRRs are considered as AESIs, if the event has severity of Grade 3 or higher:
IMARs
LRRs
CRS
TLS

AESIs must be reported to PureTech Health Pharmacovigilance department or its designee within the same timeframe as that which applies for SAEs.

### Assessment of Adverse Events

### Severity

All AEs are to be graded by severity using CTCAE version 5.0. If an AE is not listed in the CTCAE criteria, a corresponding grading is to be performed by the Investigator based on his/her best medical judgment as follows:
Mild (Grade 1): asymptomatic or mild symptoms; clinical or diagnostic observations only; intervention not indicated
Moderate (Grade 2): minimal, local, or noninvasive intervention indicated; limited age appropriate instrumental activities of daily living (ADL)
Severe (Grade 3): medically significant but not immediately life-threatening; hospitalization or prolongation of hospitalization indicated; disabling; or limiting self care ADL
Life-threatening (Grade 4): life-threatening consequences; urgent intervention indicated
Death (Grade 5): death related to an AE

The terms “severe” and “serious” are not synonymous. Severity is a measure of intensity (as characterized above) whereas seriousness as defined herein, defines the requirements for reporting obligations.

### VIII. Statistics

### Sample Size

This dose escalation study will establish either the MTD and/or the minimum safe and effective biological dose to determine the RP2D dose or doses for G9.2-17(IgG4) using a 4+2 algorithm-based does-escalation design. With the potential inclusion of patient backfill, it is anticipated that a total sample size of up to 50 patients are to be enrolled into the study.

### Analysis Populations

The intent-to-treat (ITT) population will be defined as those patients who received at least one dose of G9.2-17(IgG4). The efficacy analyses will be performed for the ITT. Patient disposition will be performed for the ITT. The Efficacy population will be defined as all patients in the ITT and having at least one measurable overall response at 1 month. This population is to be used for a sensitivity analysis.

The per protocol (PP) population includes any patient who received at least one full cycle of G9.2-17(IgG4) and without major protocol deviations.

The safety population (SAF) is defined as all patients who receive at least one dose of G9.2-17(IgG4). The safety analyses will be performed for the SAF.

The PK population includes any patients who received as least one dose of G9.2-17(IgG4) and have at least one evaluable post-dose concentration.

The pharmacodynamics (PD) population is defined as those patients who have received at least one full cycle of G9.2-17(IgG4), and who have and at least one post-treatment PD assessment along with a baseline PD assessment, respectively.

### General Statistical Plan

A database lock and primary analysis is performed after the last patient has had their primary endpoint event. A final study analysis is performed after study completion. All analyses are descriptive.

Unless otherwise specified, all continuous endpoints are summarized using descriptive statistics, which includes the number of patients (n), mean, standard deviation, median, minimum, and maximum. All categorical endpoints are summarized using frequencies and percentages, with 95% CI. The screening measurement is the last value on or before the date of first study treatment. Survival curves for, PFS and OS are generated by the method of Kaplan-Meier from the day treatment was initiated, but there is no comparative analysis between study arms in any part of the trial. Waterfall and Swimmers plots are used to graphically present the ORR and DoR for all patients in each study arm, within each tumor type evaluated in Part 2. All statistical analyses are performed using SAS®, version 9.2. (SAS, Cary, NC).

### Safety Analysis

All safety analyses are made on the SAF unless otherwise specified and are analyzed using descriptive statistics

### Efficacy Analysis

Disease response assessed is summarized descriptively for the ITT and PP. A sensitivity analysis is performed using the Efficacy Populations.

### Pharmacokinetics, Pharmacodynamics, and Immunogenicity

PK, PD, and immunogenicity are summarized descriptively for the PK/PD population.

G9.2-17 (IgG4) has a quick clearance rate in human subjects as compared with conventional antibody therapeutics. Once the safety of the anti-Gal-9 antibody was established, a treatment regimen that comprises a dosing schedule of once very week was developed to maintain a consistent systemic exposure level of the anti-Galectin 9 antibody.

The therapeutic methods provided herein, comprising administering an anti-Galectin-9 antibody such as G9.2-17(IgG4) at the specific dosage and dosing schedules provided herein (e.g., those used in the clinical trial provided herein), are expected to yield one or more positive clinical outcome(s), for example, reducing tumor size, delaying tumor growth, alleviating lesions and/or delaying development of lesions, and/or elongating survival of human patients having the target hematological malignancies such as AML, MDS, or ALL (T cell or B cell ALL), which may be refractory and/or relapsed.

### Example 2: Analysis of AML Patients Treated by Anti-Galectin 9 Antibody

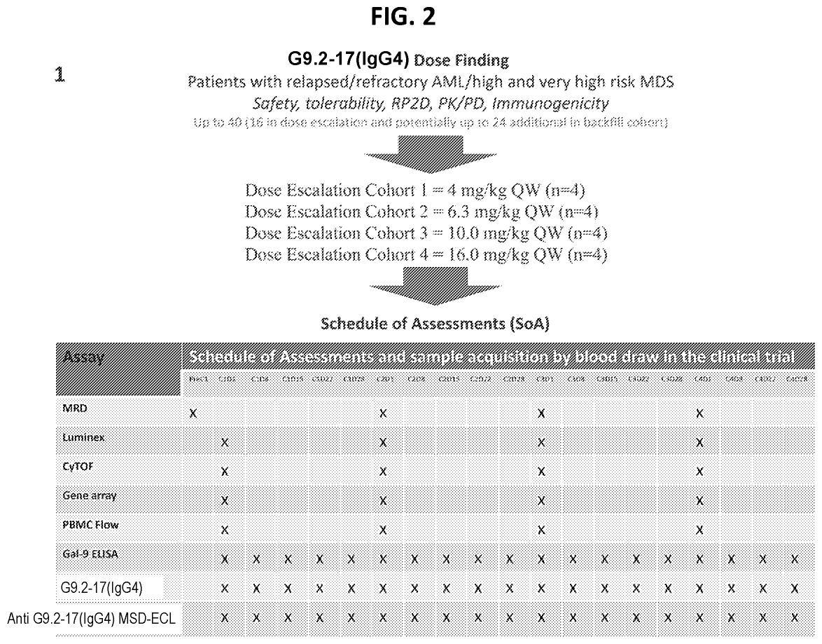

Biological samples such as blood samples are to be collected from the AML patients who participate in the clinical trial described in Example 1 above. Pharmacokinetic (PK), immunogenicity and pharmacodynamic (PD) profile of the anti-galectin 9 antibody (G9.2-17(IgG4) are investigated for studying immunological and molecular changes in the peripheral blood and bone marrow. The clinical trial schedule of assessments is provided in FIG. 2.

The following assays are to be used in assessing PD profile of the anti-galectin 9 antibody.

### Analytical Assays:

1. Luminex
2. Immunophenotyping PBMC flow
3. CyTOF (Cytometry by time of flight)
4. MRD assessment by PCR
5. Gene array: Advanta Immuno-Oncology Gene Expression Assay
6. Total G9.2-17(IgG4) (PK) LC/MS/MS analysis—already being usen I the G9.2-17(IgG4) solid tumor study
7. Anti G9.2-17(IgG4) MSD-ECL—already being usen I the G9.2-17(IgG4) solid tumor study
8. Galectin-9 serum ELISA—already being usen I the G9.2-17(IgG4) solid tumor study

Total G9.2-17(IgG4) (PK) LC/MS/MS analysis, Anti G9.2-17(IgG4) MSD-ECL and Galectin-9 serum ELISA are already being done in the G9.2-17(IgG4) solid tumor trial at PPD Central Laboratory with developed and validated methods.

Luminex: Luminex is an important tool in the interrogation of the cytokine milieu in physiologic and pathologic processes because it enables patterns of cytokines to be evaluated, thereby providing a more comprehensive depiction of the complex cell signaling network than was previously possible. It provides more robust data delivery with higher throughput and density than traditional methods, but with accuracy comparable to that of ELISA. Luminex requires significantly lower sample volumes and shorter assay times than ELISA and has revolutionized our ability to rapidly assess the relative concentration of various soluble factors. Luminex is unique in that it offers both high-density (multiplexing 100 tests per sample) and high throughput (can test up to 1,000 samples per day) simultaneously. It facilitates the simultaneous evaluation of multiple immune mediators with advantages of higher throughput, smaller sample volume, and lower cost. Markers to look at are: IFN-γ, IL-10, IL-12p70, IL-13, IL-1β, IL-2, IL-4, IL-6, IL-8, TNF-α, MIP 1b, MCP-1, MIP 1a, IL-17a, IL-5, TGF-β.

Immunophenotyping PBMC flow: Multi parameter flow cytometry technology is to be used for comprehensive monitoring of immune responses to immunotherapy from a single tube of blood, maximizing information from often limited patient material. Immune markers encompassed in the PBMC immunophenotyping are non-overlapping with the ones in the CyTOF panel and include molecules such as gal-9, TIM-3, TIGIT, PD-1, PD-L1, CTLA-4, CD47, CD73, CD123, KI67, in addition to standard immune cell class markers (e.g., CD3, CD4, CD8, CD11b/c CD14, CD15, CD16, CD19, CD29, CD25, CD27, CD28, CD33, CD38, CD45, CD45RO, CD45RA, CD56, CD57, CD66b, CD95, CD127, CD161, CD163, CD294, FOXP3, CCR4, CCR6, CXCR3, CXCR5, CXCR7, TCRγδ, HLA-DR, IgD, Ki67, TIGIT, Lag-3, and PD-1), which allow for stratifying the expression of the immune checkpoint molecules and other immune targets in AML, across these immune cell populations, so that their potential change/modulation dynamically can be tracked, upon exposure to the anti-galectin 9 antibody. Granulocytes are to be excluded as PBMCs, not the whole blood, are to be analyzed. Analyzing these markers by flow may also indicate potential combination therapies that may be valuable to explore clinically with G9.2-17(IgG4), and some, e.g., anti-CD47, anti-CD123, anti-TIGIT, anti-CD73, anti-TIM-3, are already in clinical trials.

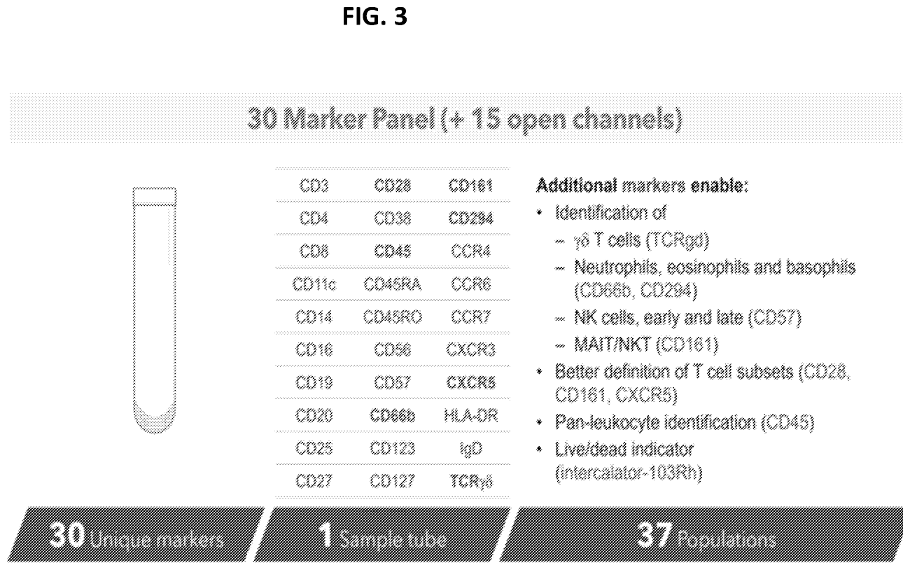

CyTOF: Cytometry by time of flight, is an application of mass cytometry used to quantify labeled targets on the surface and interior of single cells. CyTOF utilize low cell numbers and antibodies conjugated to rare heavy metal isotopes that are not normally present in biological specimens. CyTOF allows the quantification of multiple cellular components simultaneously using an ICP-MS detector. Clinical trial samples are to be processed using the most comprehensive Maxpar® Human Immune Monitoring Panel, containing 30 intracellular and extracellular parameters/markers, which allows for highly multiplexed analyses of patient samples, reliably quantify 37 immune cell populations in human whole blood or PBMC. As clinical trial samples are precious, this method is selected because it requires only 270 μl of whole blood. This method is to be used because flow cytometry is limited by the number of parameters that can be simultaneously analyzed, severely restricting its utility. Markers that are to be examined are provided in FIG. 3. Additional markers to examine are provided above.

MRD assessment by RT-qPCR: Monitoring of minimal residual disease (MRD) is a routine clinical practice in hematological cancers. The MRD techniques need to be sensitive broadly applicable, accurate, reliable, fast, and affordable. MRD serves several important functions. Clinicians use this prognostic marker to evaluate response to treatment, predict the likelihood of relapse, and identify if a patient is in remission or suffering a relapse. The sensitivity of a test dictates its effectiveness at detecting MRD, and the primary outcome is the MRD cellular level. The cutoff level is 0.01% MRD cells, or 1 MRD cell per 10,000 cells.1 Measurements above this indicate a higher risk of relapse compared with results below 0.01%, and as the numbers increase (e.g., MRD >1%), so does the risk of relapse, while chances of survival drop. Thus far, flow cytometry and polymerase chain reaction (PCR) analysis are the preferred methods. In the clinical trial, MRD assessment by flow cytometry is executed by local site laboratories, while samples are sent for PCR analyses. Peripheral blood can be used for both methods, but the MRD levels would be 10 times lower compared with bone marrow. Therefore, bone marrow samples are collected as well whenever possible. PCR has a one-log-higher sensitivity compared with flowcytometry, and that is why this modality is to be performed to measure MRD in addition to flow cytometry.

Gene array: Immuno-Oncology Gene Expression Assay for Translational and Clinical Research consists of 170 highly informative genes involved in immune cell identification, immune and cancer cell function, immune regulation and cell fate, and checkpoint inhibitor (hence immunotherapy agent) response. It has been designed for use with the Biomark™ HD system, the Advanta Immuno-Oncology Gene Expression Assay sensitively detects these gene expression markers which get analyzed across defined cell subsets. This assay can enable and accelerate the development of immunotherapies by identifying predictive biomarker signatures for therapeutic response. Tumor gene expression profiling has proven effective in measuring immune response during cancer progression and therapeutic response. Previous approaches using large, preconfigured panels containing hundreds of genes or transcriptome analysis, however, are both costly and time-consuming. Large, preconfigured panels can also be difficult to customize for specific experimental needs. The Advanta Immuno-Oncology Gene Expression Assay was developed in collaboration with leading researchers from academia and biopharma to provide the right balance of biomarker breadth, assay flexibility and workflow efficiency. The Advanta Immuno-Oncology Gene Expression Assay is available as a two-panel set. The first panel includes 91 key markers of tumor immune response that were previously shown in a multicenter international clinical trial to inform tumor progression and immunotherapy response. The second panel includes 74 additional highly informative immuno-oncology markers and 17 open assay inlets for additional customization. Both panels contain the same five reference genes. Combined with Fluidigm microfluidics technology, this Advanta assay uniquely offers significant workflow efficiencies over traditional gene expression profiling methods. Each reaction is miniaturized to nanoliter volume, meaning that clinical trial blood samples would be used in the most efficient manner and controlled using precise automation to empower accurate and cost-effective qPCR testing across a large dynamic range.

Galectin-9 ELISA: Patient blood are collected following routine practice, and serum isolated for processing on the Boster Picokine™ human galectin-9 pre-coated Enzyme-Linked Immunosorbent Assay (ELISA) kit, at the designated central laboratory (Flagship Biosciences). This is a solid phase immunoassay specially designed to measure human galectin-9 with a 96-well strip plate. The detection antibody is a biotinylated antibody specific for gal-9. The capture antibody is monoclonal antibody from mouse and the detection antibody is polyclonal antibody from goat. Optical density of the wells is measured with FLUOstar Optima microplate reader (BMG Labtech). Serum galectin-9 concentrations are calculated with BMG Optima 2.10 R2 software.

The anti-galectin 9 antibody (G9.2-17(IgG4)) may reduce leukemia cell burden in the bone marrow and the periphery, and at the very least reactivate effector T cells and macrophages and broadly modulate the ratio and phenotype of immune cell populations and/or cytokines in the blood, to reflect the reversal of immunosuppression that exists in AML/MDS. The anti-galectin 9 antibody may also lower galectin-9 serum levels, where it is elevated at baseline. Research steps outlined here are designed to move efficiently during Part 1 of the clinical trial and may inform and potentially accelerate further clinical testing in Part 2. In accordance with this vision, the plan is centered on timely sample acquisition, analyses and reporting, while collecting safety and any preliminary efficacy readouts in the treated patients. Clinical trials can be lengthy and costly, and any unnecessary exposure of non-responders to ineffective immune modulating drugs should be avoided. In practical terms, one goal of this study is to identify and develop assay(s), as complementary diagnostics, to determine the probability that an individual patient may respond to treatment of the anti-galectin9 antibody and reflect its anti-leukemic and immunomodulatory effects in vivo. This may enable effective selection of likely responders and accelerate further clinical development of the antibody.

The anti-galectin 9 antibody may have a rapid impact (within the first week post administration) on peripheral immunodynamics and circulating target levels, which is why on-study sampling is applied to cover early and late timepoints. Equally many blood collection time points coincide with study PK sampling, which may allow for deriving correlations between PK parameters and any immunomodulatory signals detected through phenotyping and galectin-9 serum levels.

This study elucidates immunological pathways and treatment responses while ensuring that: i) the planned assays measure the endpoints desired and encompass both phenotypic and functional immune markers; ii) assays that address the relevant stimulatory and regulatory pathways are considered; iii) both host- and tumor-relevant markers that may impact on outcomes are measured; iv) the assays are reproducible with acceptable variation; v) methods for quality assurance and quality control are in place; vi) central laboratory is validated; vii) the compartment in which the assay needs to be measured (peripheral blood/bone marrow) is carefully considered; viii) timepoints of sample acquisition during the treatment regimen at which the assays are conducted are selected to best determine dynamics of the effect; ix) the analysis using the appropriate statistical and bioinformatics methods are planned; and x) specimens are banked if possible (cells, serum, plasma, bone marrow) for future assays (functional, phenotypic, DNA and mRNA).

NanoString Assay: Patient blood samples are collected and PBMCs are isolated following routine practice. RNA are extracted from the PBMC samples using the RNeasy Mini Kit (WI-84), an elution volume of 30 μL are to be used. Subsequently, the RNA concentration are measured using a fluorometric analysis method, either with a Quant-iT RiboGreen RNA Reagent Kit (WI-82) or Qubit RNA assay kit (WI-258). The optimal concentration of the samples is 60 ng/μL in a 5 μL volume. The following nCounter gene expression panel is used in this study: Human nCounter® PanCancer 10360 panel with RLF: NS_IO_360_v1.0. The nCounter gene expression analysis is performed in batches of maximum 11 clinical samples. In addition, every run also contains a panel standard sample for the respective nCounter assay.

For data analysis, raw data is normalized using background correction, normalization using the spiked-in positive controls and normalization using the housekeeping genes.

### EQUIVALENTS

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

While several inventive embodiments have been described and illustrated herein, those of ordinary skill in the art are readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art are readily appreciate that all parameters, dimensions, materials, and configurations described herein are meant to be exemplary and that the actual parameters, dimensions, materials, and/or configurations depend upon the specific application or applications for which the inventive teachings is/are used. Those skilled in the art are recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” are refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for treating a hematologic malignancy, comprising administering to a subject in need thereof a pharmaceutical composition comprising an antibody that binds human galectin-9 (anti-Gal9 antibody), wherein the anti-Gal9 antibody is administered to the subject at a dose of about 2 mg/kg to about 32 mg/kg once every week to once every 4 weeks.

2. The method of claim 1, wherein the anti-Gal9 antibody comprises:
(a) a light chain comprising a light chain variable region (V_{L}), which comprises a light chain (LC) complementarity determining region 1 (CDR1) comprising the amino acid sequence of SEQ ID NO: 1, a LC complementarity determining region 2 (CDR2) comprising the amino acid sequence of SEQ ID NO: 2, and a LC complementarity determining region 3 (CDR3) comprising the amino acid sequence of SEQ ID NO: 3 and
(b) a heavy chain comprising a heavy chain variable region (V_{H}), which comprises a heavy chain (HC) complementarity determining region 1 (CDR1) comprising the amino acid sequence of SEQ ID NO: 4, a HC complementarity determining region 2 (CDR2) comprising the amino acid sequence of SEQ ID NO: 5, and a HC complementarity determining region 3 (CDR3) comprising the amino acid sequence of SEQ ID NO: 6.

3. The method of claim 2, wherein the anti-Gal9 antibody comprises the V_{L}, which comprises the amino acid sequence of SEQ ID NO:8, and the V_{H}, which comprises the amino acid sequence of SEQ ID NO: 7.

4. The method of claim 1, wherein the anti-Gal9 antibody is an IgG1 molecule or an IgG4 molecule.

5. The method of claim 4, wherein the anti-Gal9 antibody is an IgG4 molecule comprising a modified Fc region relative to the wild-type human IgG4 counterpart.

6. The method of claim 5, wherein the modified Fc region comprises the amino acid sequence of SEQ ID NO: 14.

7. The method of claim 6, wherein the anti-Gal9 antibody comprises a heavy chain, which comprises the amino acid sequence of SEQ ID NO: 19, and a light chain, which comprises the amino acid sequence of SEQ ID NO: 15.

8. The method of claim 1, wherein the anti-Gal9 antibody is administered to the subject at a dose of about 2 mg/kg, about 4 mg/kg, about 6.3 mg/kg, about 7.5 mg/kg, about 10 mg/kg, about 12 mg/kg, or about 16 mg/kg.

9. The method of claim 1, wherein the anti-Gal9 antibody is administered to the subject at a dose of about 2 mg/kg, about 4 mg/kg, about 7.5 mg/kg, about 12 mg/kg, or about 16 mg/kg.

10. The method of claim 8, wherein the anti-Gal9 antibody is administered once every two weeks.

11. The method of claim 8, wherein the anti-Gal9 antibody is administered once every week.

12. The method of claim 1, wherein the anti-Gal9 antibody is administered at a dose of 4.0 mg/kg every week, at a dose of 6.3 mg/kg every week, at a dose of 7.5 mg/kg every week, at a dose of 10.0 mg/kg every week, at a dose of 12 mg/kg every week, or at a dose of 16.0 mg/kg every week.

13. The method of claim 1, wherein the anti-Gal9 antibody is administered at a dose of 4.0 mg/kg every week, at a dose of 7.5 mg/kg every week, at a dose of 12 mg/kg every week, or at a dose of 16.0 mg/kg every week.

14. The method of claim 1, wherein the subject is a human patient having a leukemia or lymphoma.

15. The method of claim 14, wherein the human patient has acute myeloid leukemia (AML), myelodysplastic syndromes (MDS), or acute lymphoblastic leukemia (ALL).

16. The method of claim 1, wherein the hematologic malignancy is refractory and/or relapsed.

17. The method of claim 16, wherein the hematologic malignancy is refractory and/or relapsed and high-risk MDS.

18. The method of claim 15, wherein the human patient has relapsed and/or refractory AML post at least one line of prior therapy.

19. The method of claim 15, wherein the human patient has relapsed and/or refractory MDS post at least one line of prior therapy.

20. The method of claim 1, wherein the human patient has undergone or is undergoing a chemotherapy for treating the hematologic malignancy.

21. The method of claim 1, which comprises at least one cycle of treatment that consists of 28 days, during which the anti-Gal9 antibody is administered to the subject once every week.

22. The method of claim 1, wherein the method comprises the anti-Gal9 antibody as the only active agent for treating the hematologic malignancy.

23. The method of claim 1, further comprising monitoring occurrence of an adverse event in the course of the treatment.

24. The method of claim 21, further comprising reducing the dose of the anti-Gal9 antibody in the subject who has occurrence of the adverse event occurs.


SEQUENCE LISTING (35 sequences)

SEQ ID NO: 1 - protein, 11 aa, synthetic construct
RASQSVSSAVA

SEQ ID NO: 2 - protein, 7 aa, synthetic construct
SASSLYS

SEQ ID NO: 3 - protein, 9 aa, synthetic construct
QQSSTDPIT

SEQ ID NO: 4 - protein, 9 aa, synthetic construct
FTVSSSSIH

SEQ ID NO: 5 - protein, 17 aa, synthetic construct
YISSSSGYTYYADSVKG

SEQ ID NO: 6 - protein, 15 aa, synthetic construct
YWSYPSWWPYRGMDY

SEQ ID NO: 7 - protein, 124 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSS

SEQ ID NO: 8 - protein, 108 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSSTDPITFGQGTKVEIKR

SEQ ID NO: 9 - protein, 20 aa, synthetic construct
MYRMQLLSCIALSLALVTNS

SEQ ID NO: 10 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 14 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 15 - protein, 214 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSSTDPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 454 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 454 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 451 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 19 - protein, 451 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 20 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 451 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 451 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 27 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 28 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 29 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 30 - protein, 453 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 453 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 450 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 33 - protein, 450 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 34 - protein, 450 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 35 - protein, 450 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG